(19) United States
(12) Patent Application Publication
(10) Pub. No.: US 2021/0225456 A1
(43) Pub. Date: Jul. 22, 2021

(54) METHOD FOR DETECTING GENETIC VARIATION IN HIGHLY HOMOLOGOUS SEQUENCES BY INDEPENDENT ALIGNMENT AND PAIRING OF SEQUENCE READS

(71) Applicant: MYRIAD WOMEN'S HEALTH, INC., South San Francisco, CA (US)

(72) Inventors: Peter GRAUMAN, South San Francisco, CA (US); Genevieve GOULD, South San Francisco, CA (US); Dale MUZZEY, South San Francisco, CA (US)

(73) Assignee: MYRIAD WOMEN'S HEALTH, INC., South San Francisco, CA (US)

(21) Appl. No.: 17/158,978

(22) Filed: Jan. 26, 2021

Related U.S. Application Data
(63) Continuation of application No. PCT/US2019/043678, filed on Jul. 26, 2019.
(60) Provisional application No. 62/711,454, filed on Jul. 27, 2018.
(60) Provisional application No. 62/730,479, filed on Sep. 12, 2018.

(30) Foreign Application Priority Data
Jul. 26, 2019 (US) PCT/US2019/043678

(51) Int. Cl. G16B 30/10; G16B 20/20; C12Q 1/6844; G16B 20/10; G16B 40/30

(52) U.S. Cl. CPC G16B 30/10; G16B 20/20; G16B 40/30; G16B 20/10; C12Q 1/6844

(57) ABSTRACT

The method described herein combines experimental and analytical approaches to resolve the structure of a genomic region in the genome of a subject whose sequence is highly homologous to one or more other regions of the genome. For example, the genomic region may be a gene and the highly homologous other region may be a pseudogene. The method involves independent alignment, pairing, and analysis of sequence reads from the genomic region and the highly homologous other region to identify genetic variation. Also described herein is a computer-assisted method for such methods.

### CROSS-REFERENCE TO RELATED APPLICATIONS

The present application is a continuation of International Patent Application No. PCT/US2019/043678, filed Jul. 26, 2019, which claims priority to U.S. Provisional Application No. 62/711,454, filed Jul. 27, 2018, and to U.S. Provisional Application No. 62/730,479, filed Sep. 12, 2018, each of which is hereby incorporated in its entirety including all tables, figures, and claims.

### TECHNICAL FIELD

The following disclosure relates generally to determining genetic variation, more specifically, to determining genetic variation in highly homologous regions of interest in a genome, for example, in genomic regions comprising a gene and a pseudogene.

### BACKGROUND

Individual genomic variants inherited through the germline account for approximately 5% to 10% percent of cancer [1-3]. This heritable component can increase risk for malignancies across a range of tissues [4,5]—such as breast, colorectal, pancreatic, and prostate—and is associated with pathogenic variants in >100 genes [6]. To assess patients' risk for such cancers, hereditary cancer screening (HCS) typically uses targeted next-generation sequencing (NGS) to detect relevant variants in the coding regions and select noncoding regions on a multigene testing panel.

In most genomic regions interrogated by HCS panels, NGS alone is sufficient to yield high sensitivity and specificity [7,8]; high accuracy is critical for HCS because test results prompt patients to alter their clinical-management decisions [9,10]. In a minority of regions, however, standard NGS strategies that use hybridization to capture and sequence short DNA fragments could incorrectly identify genotypes. Genes that pose particular challenges often have homologous sequences (e.g., pseudogenes) elsewhere in the genome that are captured and sequenced along with the gene itself, complicating alignment and the identification of variants specific to the gene.

Thus, there remains a need for improved methods of detection of genetic variation in homologous regions of the genome.

### BRIEF SUMMARY OF THE INVENTION

Current technologies that allow determination of genotypes for highly homologous genes and the corresponding homologs are time- and labor-intensive, as well as expensive, making them unsuitable for widespread clinical use.

The presently disclosed methods may be practiced in an affordable and high-throughput manner. Thus, there are significant time, labor and expense savings. In addition, the present method overcomes the problem of resolving structure/copy-number/genotype in regions where the unique alignment of NGS reads to genes or their homologs is compromised.

In an aspect there is provided herein a method for determining the genomic structure (i.e., genotype) of an individual with respect to a gene of interest, wherein the gene of interest has a highly homologous homolog, for example a pseudogene.

In one embodiment, a method is provided for detecting genetic variation in a genome of a subject, the genome comprising highly homologous first and second regions of interest, the method comprising: (a) obtaining sequence reads by paired-end sequencing from multiple sites of interest in the first and second regions of interest, wherein the sequence reads comprise a first read and a second read obtained at each site of interest; (b) aligning sequence reads to a reference genome, wherein first reads and second reads are aligned to the reference genome separately and the aligner emits multiple possible alignments for each of the first and second reads; (c) identifying first reads and second reads that align to the first region of interest; (d) pairing a first read and a second read from the reads identified in step (c), thereby generating a top paired alignment; and (e) detecting the genetic variation in the top paired alignment generated in step (d). In another embodiment, the method comprises, before step (b), aligning first reads and second reads to a reference genome, wherein the aligner emits the best possible paired-end alignment to the first or second region of interest for each pair of first and second reads, and wherein only paired-end reads associated with a top alignment score to the first or second regions of interest are aligned separately in step (b). In one embodiment, the reference genome does not comprise a masked or modified portion of a first or second homologous region of interest. In one embodiment, the method is computer-implemented.

In one embodiment, a method is provided for detecting genetic variation in a genome of a subject, the genome comprising highly homologous first and second regions of interest, the method comprising obtaining sequence reads by paired-end sequencing from multiple sites of interest in the first and second regions of interest, wherein the sequence reads comprise a first read and a second read obtained at each site of interest, wherein the sequence reads are obtained by direct targeted sequencing (DTS) of the multiple sites of interest, and wherein the first read comprises a genomic sequence read and the second read comprises a probe sequence read associated with a site of interest.

In one embodiment, a method is provided for detecting genetic variation in a genome of a subject, the genome comprising highly homologous first and second regions of interest, the method comprising: (a) obtaining sequence reads by paired-end sequencing from multiple sites of interest in the first and second regions of interest, wherein the sequence reads comprise a first read and a second read obtained at each site of interest; (b) aligning sequence reads to a reference genome, wherein first reads and second reads are aligned to the reference genome separately and the aligner emits multiple possible alignments for each of the first and second reads; (c) identifying first reads and second reads that align to the first region of interest; (d) pairing a first read and a second read from the reads identified in step (c), thereby generating a top paired alignment; and (e) detecting the genetic variation in the top paired alignment generated in step (d). In one embodiment, the sequence reads are aligned using the Burrows-Wheeler Aligner (BWA) algorithm. In one embodiment, the aligner only emits alignments that meet a minimum alignment score for the first and second regions of interest. In one embodiment, a first read and a second read are paired to generate a top paired alignment only if the alignments of the first read and the second read to the first region of interest are within a certain number of bases of each other. In one embodiment, a first read and a second read are paired to generate a top paired alignment only if the alignments of the first read and the second read to the first region of interest are within about 100 bp, about 200 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp, about 1500 bp, or more than 1500 bp.

In one embodiment, a method is provided for detecting genetic variation in a genome of a subject, the genome comprising highly homologous first and second regions of interest, the method comprising: (a) obtaining sequence reads by paired-end sequencing from multiple sites of interest in the first and second regions of interest, wherein the sequence reads comprise a first read and a second read obtained at each site of interest; (b) aligning sequence reads to a reference genome, wherein first reads and second reads are aligned to the reference genome separately and the aligner emits multiple possible alignments for each of the first and second reads; (c) identifying first reads and second reads that align to the first region of interest; (d) pairing a first read and a second read from the reads identified in step (c), thereby generating a top paired alignment; and (e) detecting the genetic variation in the top paired alignment generated in step (d). In one embodiment, the method comprises generating multiple paired alignments in step (d), calculating an alignment score for each of the multiple paired alignments, and identifying the top paired alignment as having the highest alignment score. In one embodiment, the top paired alignment in step (d) is selected as having the smallest template length.

In one embodiment, a method is provided for detecting genetic variation in a genome of a subject, the genome comprising highly homologous first and second regions of interest, the method comprising: (a) obtaining sequence reads by paired-end sequencing from multiple sites of interest in the first and second regions of interest, wherein the sequence reads comprise a first read and a second read obtained at each site of interest; (b) aligning sequence reads to a reference genome, wherein first reads and second reads are aligned to the reference genome separately and the aligner emits multiple possible alignments for each of the first and second reads; (c) identifying first reads and second reads that align to the first region of interest; (d) pairing a first read and a second read from the reads identified in step (c), thereby generating a top paired alignment; and (e) detecting the genetic variation in the top paired alignment generated in step (d). In one embodiment, the genetic variation comprises SNPs, indels, inversions, and/or CNVs. In one embodiment, the detecting in step (e) comprises calling SNPs, indels, inversions, and/or CNVs. In one embodiment, the detecting in step (e) comprises using a hidden Markov model (HMM) caller to determine a copy number. In one embodiment, the detecting in step (e) is based on an expected ploidy of 2. In one embodiment, the detecting in step (e) is based on an expected ploidy of 4. In one embodiment, if a genetic variation is detected in step (e), a portion of the subject's genome is amplified by long-range PCR and assayed by multiplex ligation-dependent probe amplification (MLPA). In one embodiment, if a genetic variation is detected in step (e), a portion of the first region of interest is amplified by long-range PCR and the product or a portion thereof is sequenced by Sanger sequencing or NGS. In one embodiment, if a genetic variation is detected in step (e), the subject's genomic DNA is assayed by multiplex ligation-dependent probe amplification (MLPA).

In one embodiment, a method is provided for detecting genetic variation in a genome of a subject, the genome comprising highly homologous first and second regions of interest, the method comprising: (a) obtaining sequence reads by paired-end sequencing from multiple sites of interest in the first and second regions of interest, wherein the sequence reads comprise a first read and a second read obtained at each site of interest; (b) aligning sequence reads to a reference genome, wherein first reads and second reads are aligned to the reference genome separately and the aligner emits multiple possible alignments for each of the first and second reads; (c) identifying first reads and second reads that align to the first region of interest; (d) pairing a first read and a second read from the reads identified in step (c), thereby generating a top paired alignment; and (e) detecting the genetic variation in the top paired alignment generated in step (d). In one embodiment, the sequence reads are 30-50 bp or 100-200 bp in length. In one embodiment, the highly homologous first and second regions of interest are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more than 99% identical. In one embodiment, the sequence reads are obtained from one or more exons within the first and/or second region(s) of interest. In one embodiment, the sequence reads are obtained from one or more introns within the first and/or second region(s) of interest. In one embodiment, the sequence reads are obtained from one or more exons and introns within the first and/or second region(s) of interest. In one embodiment, the sequence reads are obtained from one or more exons and introns within the first and/or second region(s) of interest, and wherein the introns are near the exons. In one embodiment, sequence reads are obtained from one or more clinically actionable regions associated with the first and/or second region(s) of interest. In one embodiment, the first region of interest comprises a gene and the second region of interest comprises a pseudogene. In one embodiment, the first region of interest comprises a pseudogene and the second region of interest comprises a gene. In one embodiment, the first region of interest comprises two alleles. In one embodiment, the second region of interest comprises two alleles. In one embodiment, the gene is PMS2. In one embodiment, the pseudogene is PMS2CL. In one embodiment, the multiple sites of interest are within an exon of PMS2 and an exon in another part of the subject's genome. In one embodiment, the multiple sites of interest are within an exon of PMS2 and an exon of PMS2CL. In one embodiment, the multiple sites of interest are within exons 11, 12, 13, 14, and/or 15 of PMS2 and exons 2, 3, 4, 5, and/or 6 of PMS2CL. In one embodiment, the subject is a human and the sequence reads are aligned to a human reference genome.

In one embodiment, a method is provided for detecting genetic variation in a genome of a subject, the genome comprising highly homologous first and second regions of interest, the method comprising: (a) obtaining sequence reads by paired-end sequencing from multiple sites of interest in the first and second regions of interest, wherein the sequence reads comprise a first read and a second read obtained at each site of interest; (b) aligning sequence reads to a reference genome, wherein first reads and second reads are aligned to the reference genome separately and the aligner emits multiple possible alignments for each of the first and second reads; (c) identifying first reads and second reads that align to the first region of interest; (d) pairing a first read and a second read from the reads identified in step (c), thereby generating a top paired alignment; and (e) detecting the genetic variation in the top paired alignment generated in step (d). In one embodiment, a non-transitory computer-readable storage medium is provided, comprising computer-executable instructions for carrying out the methods described herein. In one embodiment, a system is provided, comprising: (a) one or more processors; (b) memory; and (c) one or more programs, wherein the one or more programs are stored in the memory and configured to be executed by the one or more processors, the one or more programs including instructions for carrying out the methods described herein.

In one embodiment, a computer system configured to execute instructions for carrying out the methods described herein is provided.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### BRIEF DESCRIPTION OF THE DRAWINGS

FIGS. 1A-1D illustrate a LR-PCR strategy for building a dataset of natural genetic variation in PMS2 and PMS2CL. FIG. 1A: Short-reads from NGS hybrid-capture data that originate from the gene (blue) and pseudogene (red) align to both the gene and pseudogene due to high homology. FIGS. 1B and 1C: Using LR-PCR that is specific to the gene or pseudogene followed by fragmentation and barcoding (FIG. 1B), the resulting short NGS reads can be assigned to the gene or pseudogene (FIG. 1C). FIG. 1D: Percent identity between the gene and pseudogene for PMS2 exons 11-15 based on the hg19 reference genome (gray) and after accounting for natural genetic variation obtained from LR-PCR samples (black).

FIGS. 2A-2B illustrate a reflex workflow for variant identification in the last exons of PMS2. FIG. 2A: Overview of sequencing and analysis workflow for the last five exons of PMS2. Colored nodes correspond to boxes in FIG. 2B. FIG. 2B: Details corresponding to workflow steps in FIG. 2A; the details of each box are described in Methods and Results. “No report” means the variant does not appear on patient reports. “Reflex” means the sample is sent for LR-PCR-based disambiguation to determine if the variant is localized to the gene or pseudogene.

FIGS. 3A-3C illustrate that a hybrid-capture and LR-PCR are concordant for SNVs and indels. FIG. 3A: Hypothetical examples to describe the concordance table for comparison of hybrid capture and LR-PCR data. All examples assume the reference base is A and the alternate (“alt”) base is T. (i) Example of a true positive (dark blue) where an alt allele is present in PMS2CL. (ii) Example of a permissible dosage error (light blue), where PMS2CL is homozygous for the alt allele but hybrid capture only calls one alt allele instead of two. (iii) Example of a false positive (light orange), where only hybrid capture detected an alt allele. (iv) Example of a false negative (dark orange), where an alt allele in PMS2CL was missed by hybrid capture. Shaded matrix on the right indicates cells that represent true positives, permissible dosage errors, false positives, and false negatives. Numbers on axes denote the total number of alt alleles in either the hybrid capture data or the PMS2/PMS2CL LR-PCR data. FIG. 3B: Diploid SNV and indel concordance for exon 11 of PMS2. Numbers on axes denote the number of alt alleles where 0 is equivalent to 0/0, 1 is equivalent to 0/1, and 2 is equivalent to 1/1. 95% confidence intervals in brackets. FIG. 3C: Four-copy SNV and indel concordance for exons 12-15 of PMS2/PMS2CL, as explained in FIG. 3A.

FIGS. 4A-4B illustrate that simulated indels increase confidence in indel sensitivity. FIG. 4A: Schematic of simulating a tetraploid indel by combining sequencing data from two diploid samples. FIG. 4B: Results of tetraploid indel simulations in the same format as FIG. 3A.

FIGS. 5A-5D illustrate that Hybrid capture, LR-PCR, and MLPA are concordant for CNVs. FIG. 5A: All CNVs called in the hybrid capture data and corresponding orthogonal confirmation data. FIG. 5B: Hybrid capture data for the patient sample with an exon 13-14 deletion depicts copy-number estimates across the locus (bins). Gray regions denote the last four exons of PMS2. White regions denote introns. Yellow box indicates region of the CNV call. FIG. 5C: MLPA data for the exon 13-14 deletion patient sample. PMS2-specific (solid blue), PMS2CL-specific (solid red), and PMS2/PMS2CL degenerate MLPA probes (blue and red stripes) show the deletion in exons 13-14 of PMS2CL. FIG. 5D: LR-PCR data for the exon 13-14 deletion sample depicting copy number estimates across the locus (bins) for PMS2 (blue, top) and PMS2CL (red, bottom). Gray regions depict exons 11-15 of PMS2 and white regions depict introns as in FIG. 5B.

FIG. 6 illustrates orthogonal datasets used to build a hybrid capture assay. As shown, FIG. 6 is a diagram demonstrating the assays, datasets, algorithms, and analyses used to build the hybrid capture assay for the last five exons of PMS2. The Coriell samples (1b) can be used by other researchers without repeating the LR-PCR as provided in accession #PRJEB27948. Genomic DNA (gDNA).

FIGS. 7A-7C illustrate that PMS2 exons 11-15 reference genotypes (from Polaris and GIAB) are inconsistent with PMS2 LR-PCR. FIG. 7A: Concordance between LR-PCR variant calls and Polaris variant calls. FIG. 7B: Concordance between LR-PCR variant calls and the GIAB multisample call set (including high confidence and filtered variant calls) for all five GIAB samples. FIG. 7C: Concordance between LR-PCR variant calls and the 10× Genomics haplotype call set available for four GIAB samples.

FIGS. 8A-8B illustrate that RNA data corroborate hybrid capture and LR-PCR data. FIG. 8A: Concordance between hybrid capture data and RT-PCR for PMS2 and PMS2CL. FIG. 8B: Concordance between hybrid capture data and LR-PCR for PMS2 and PMS2CL.

FIG. 9 is a chart illustrating an embodiment of the method described herein comprising “ambiguous alignment” of first and second DTS reads from a region of interest.

FIG. 10 is a diagram illustrating an exemplary system and environment in which various embodiments of the invention may operate.

FIG. 11 is a diagram illustrating an exemplary computing system.

The file of this patent contains at least one drawing in color. Copies of this patent or patent publication with color drawing(s) will be provided by the Office upon request and payment of the necessary fee.

### DETAILED DESCRIPTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this invention. Although any methods and materials similar or equivalent to those described herein can be used in the practice or testing of the present invention, the preferred methods and materials are described. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Numeric ranges are inclusive of the numbers defining the range. The term “about” is used herein to mean plus or minus ten percent (10%) of a value. For example, “about 100” refers to any number between 90 and 110.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

Supplementary data, including any tables referenced (e.g., Table S1, Table S2, etc.), will be made available upon request. A version of the scientific manuscript related to this patent application is provided herewith as an Appendix.

### I. Definitions

As used herein, “purified” and its derivatives, means that a molecule is present in a sample at a concentration of at least 90% by weight, 95% by weight, or at least 98% by weight of the sample in which it is contained.

The term “isolated” and its derivatives as used herein refers to a molecule that is separated from at least one other molecule with which it is ordinarily associated, for example, in its natural environment. An isolated nucleic acid molecule includes a nucleic acid molecule originally contained in cells that ordinarily express the nucleic acid molecule, but the nucleic acid molecule is present extrachromasomally or at a chromosomal location that is different from its natural chromosomal location.

The term “% identity” and its derivatives are used interchangeably herein with the term “% homology” and its derivatives to refer to the level of a nucleic acid or an amino acid sequence's identity between another nucleic acid sequence or any other polypeptides, or the polypeptide's amino acid sequence, where the sequences are aligned using a sequence alignment program, for example, using the Basic Local Alignment Search Tool algorithm. In the case of a nucleic acid the term also applies to the intronic and/or intergenic regions.

For example, as used herein, 80% homology means the same thing as 80% sequence identity determined by a defined algorithm, and accordingly a homolog or a highly homologous sequence of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for any one of the inventive polypeptides, as described.

As used herein, “highly homologous” and its derivatives mean that the % homology or % identity between at least two different nucleotide sequences is greater than 70%. Sequences are referred to as “highly homologous” if their sequence identity is greater than 70% over a comparable length.

Exemplary computer programs which can be used to determine identity between two sequences include, but are not limited to, the suite of BLAST programs, e.g., BLASTN, BLASTX, and TBLASTX, BLASTP and TBLASTN, and BLAT publicly available on the Internet. See also, Altschul, et al., 1990 and Altschul, et al., 1997.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, S. F., et al., Nucleic Acids Res. 25:3389-3402, 1997.)

A preferred alignment of selected sequences in order to determine “% identity” between two or more sequences, is performed using for example, the CLUSTAL-W program in MacVector version 13.0.7, operated with default parameters, including an open gap penalty of 10.0, an extended gap penalty of 0.1, and a BLOSUM 30 similarity matrix.

A “sequence read” and its derivatives ranges from 30 nt to 400 nt, from 50 nt to 250 nt, from 50 nt to 150 nt, or from 100 nt to 200 nt within a nucleotide sequence.

The term “mutation” as used herein refers to both spontaneous and inherited sequence variations, including, but not limited to, variations between individuals, or between an individual's sequence and a reference sequence. Exemplary mutations include, but are not limited to, SNPs, indels (insertion or a deletion variants), copy number variants, inversions, translocations, chromosomal fusions, etc.

The term “small nucleotide polymorphism” or “SNP” and its derivatives refers to a single-nucleotide variant (SNV), a multi-nucleotide variant (MNV), or an indel variant about 100 base pairs or less.

The term “homolog” and its derivatives as used herein refer to a nucleotide sequence that is identical or nearly identical to a nucleotide sequence located elsewhere in a subject's genome. A homolog is highly homologous to a nucleotide sequence located elsewhere in a subject's genome. The homolog can be either another gene, a “pseudogene,” or a segment of sequence that is not part of a gene.

A “pseudogene” and its derivatives as used herein is a DNA sequence that closely resembles a gene in DNA sequence but harbors at least one change that renders it dysfunctional. The change may be a single residue mutation. The change may result in a splice variant. The change may result in early termination of translation. A pseudogene is a dysfunctional relative of a functional gene. Pseudogenes are characterized by a combination of homology to a known gene (i.e., a gene of interest) and nonfunctionality.

The number of pseudogenes for genes is not limited to those enumerated herein. Pseudogenes are increasingly recognized. Therefore, a person skilled in the art would be able to determine if a sequence is a pseudogene on the basis of sequence homology or by reference to a curated database such as, for example, GeneCards (genecards.org), pseudogenes.org, etc.

As used herein, a “gene of interest” and its derivatives is a gene for which determining the genotype is desired. Generally, a gene of interest has two functional copies due to the two chromosomes each having a copy of the gene of interest. The terms “gene of interest” and “gene” may be used interchangeably herein.

As used herein, a “region of interest” and its derivatives may be any region within a genome of a subject. As used herein, regions of interest generally are highly homologous sequences in the genome of a subject.

### II. Process

Samples from which polynucleotides to be analyzed by the methods described herein can be derived from multiple samples from the same individual, samples from different individuals, or combinations thereof. In some embodiments, a sample comprises a plurality of polynucleotides from a single individual. In some embodiments, a sample comprises a plurality of polynucleotides from two or more individuals. For example, the sample be derived from a pregnant woman and comprise polynucleotides from the pregnant woman and her fetus. An individual is any organism or portion thereof from which polynucleotides can be derived, non-limiting examples of which include plants, animals, fungi, protists, monerans, viruses, mitochondria, and chloroplasts. Sample polynucleotides can be isolated from a subject, such as a cell sample, tissue sample, fluid sample, or organ sample derived therefrom (or cell cultures derived from any of these), including, for example, cultured cell lines, biopsy, blood sample, cheek swab, or fluid sample containing a cell (e.g. saliva). The subject may be an animal, including but not limited to, a cow, a pig, a mouse, a rat, a chicken, a cat, a dog, etc., and is usually a mammal, such as a human. Samples can also be artificially derived, such as by chemical synthesis. In some embodiments, samples comprise DNA. In some embodiments, samples comprise cell-free DNA extracted from the plasma of a subject. In some embodiments, samples comprise genomic DNA. In some embodiments, samples comprise mitochondrial DNA, chloroplast DNA, plasmid DNA, bacterial artificial chromosomes, yeast artificial chromosomes, oligonucleotide tags, polynucleotides from an organism (e.g. bacteria, virus, or fungus) other than the subject from whom the sample is taken, or combinations thereof. In some embodiments, nucleic acids extracted comprises cell-free DNA from the maternal plasma of a pregnant woman.

Methods for the extraction and purification of nucleic acids are well known in the art. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993), with or without the use of an automated nucleic acid extractor, e.g., the Model 341 DNA Extractor available from Applied Biosystems (Foster City, Calif.); (2) stationary phase adsorption methods (U.S. Pat. No. 5,234,809; Walsh et al., 1991); and (3) salt-induced nucleic acid precipitation methods (Miller et al., (1988), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g. U.S. Pat. No. 5,705,628). In some embodiments, the above isolation methods may be preceded by an enzyme digestion step to help eliminate unwanted protein from the sample, e.g., digestion with proteinase K, or other like proteases. See, e.g., U.S. Pat. No. 7,001,724. In a preferred embodiment, the extracted DNA comprises a genome of a subject.

In some embodiments, a library comprising a plurality of nucleic acid molecules (e.g., a DNA library) is prepared form the extracted nucleic acids. In some embodiments, the nucleic acids in the plurality of nucleic acids molecules comprise an incorporated oligonucleotide, which can comprise a molecular barcode and/or one or more adapter oligonucleotides (also referred to as “adapters”).

In some embodiments, a portion of the extracted nucleic acids is amplified, such as by primer extension reactions using any suitable combination of primers and a DNA polymerase, including but not limited to polymerase chain reaction (PCR), reverse transcription, and combinations thereof. Where the template for the primer extension reaction is RNA, the product of reverse transcription is referred to as complementary DNA (cDNA). Primers useful in primer extension reactions can comprise sequences specific to one or more targets, random sequences, partially random sequences, and combinations thereof. Reaction conditions suitable for primer extension reactions are known in the art. In some embodiments, extracted DNA is amplified by long-range PCR (LR-PCR) using a specific primer, for example a gene-specific primer.

Extracted nucleic acids are sequenced. Methods for the sequencing of nucleic acids are well known in the art. In one embodiment, extracted nucleic acids are sequenced by Sanger sequencing. Extracted nucleic acids are preferably sequenced using high-throughput next-generation sequencing (NGS). In principle, any paired-end sequencing method may be used to sequence extracted DNA. In a preferred embodiment, direct targeted sequencing (DTS) is employed, wherein sequences from the region of interest are enriched, where possible, with hybrid-capture probes or PCR primers, which are designed such that the captured and sequenced fragments contain at least one sequence that distinguishes the targeted sequence from other captured sequences. In some embodiments, paired-end reads obtained by DTS of one or multiple sites of interest include a first sequence read comprising a genomic read and a second sequence read comprising a probe read associated with a site of interest in a subject's genome. In some embodiments, sequencing reads are 30-50 bp. In other embodiments, sequencing reads are 100-200 bp in length. In a preferred embodiment, sequence reads are about 40 bp. In some embodiments, DTS is used as described in U.S. Pat. No. 9,092,401, which is hereby incorporated by reference in its entirety.

For example, hybrid-capture probes may be designed to anneal adjacent to the few bases that differ between different sites of interest (“diff bases”). Where such distinguishing sequence is scarce, multiple probes may be used to capture distinguishable fragments to diminish the effect of biases inherent to each particular probe's sequence.

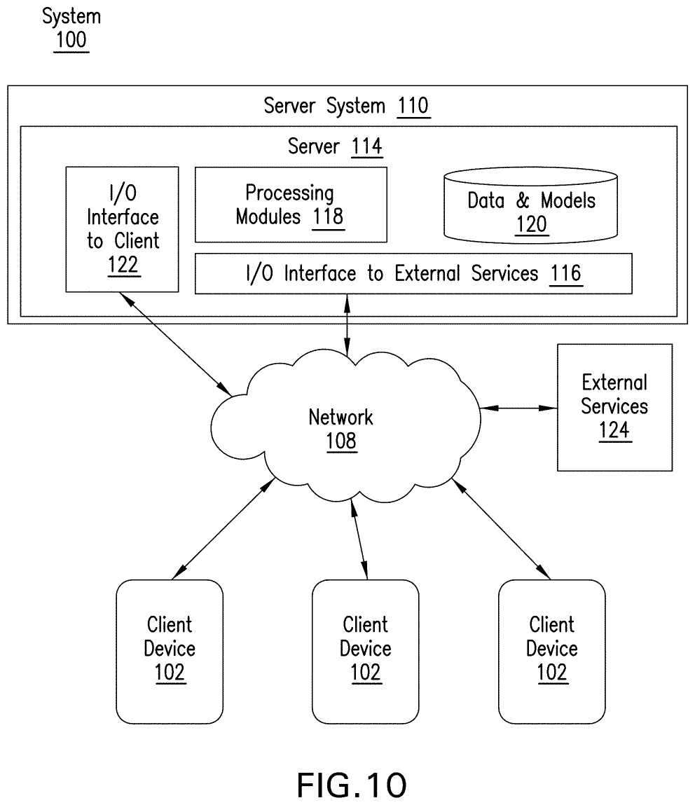

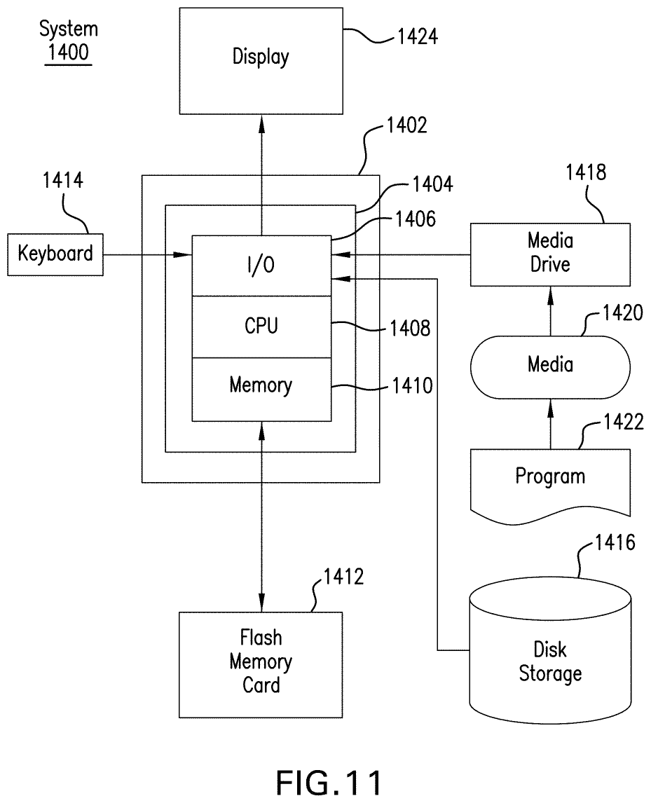

Nucleic acid sequences may be aligned to a reference genome to detect genetic variation. In a preferred embodiment, the subject is a human and the sequence reads are aligned to a human reference genome. For example, the sequence manipulation and alignment procedure (“pipeline”) may begin with raw data from a genome analyzer, for example, Genome Analyzer IIx (GAIIx) or HiSeq sequencers (Illumina; San Diego, Calif.), to infer genotypes and compute metrics from patient samples. Sequencing data from regions of interest may be generated from multiple runs of barcoded samples in a multiplexed (e.g., 12×) configuration per Flowcell lane according to a method of the invention. The sequencer raw data may include basecalls (BCL files) and various quality-control and calibration metrics. The raw basecalls and metrics may be first compiled into QSEQ files and then filtered, merged, and demultiplexed (based on barcode sequences) into sample-specific FASTQ files. FASTQ reads may be aligned to a reference genome, for example the HG19 genome, to create an initial BAM file. In some cases, each paired-end FASTQ file may be aligned to the reference genome. In other cases, each single-end FASTQ file may be separately aligned to the genome allowing for “ambiguous alignment” and reporting of the top several alignments for each read. In yet other embodiments, the overall alignment process may comprise single alignment of forward and reverse paired-end NGS reads and/or separate alignment or realignment of forward and reverse single-end NGS reads (e.g., “ambiguous alignment”). The resulting BAM file(s) may undergo several transformations to filter, clip, and refine alignments, and to recalibrate quality metrics. The final BAM file may be used to infer genotypes for known variants and to discover novel ones, producing a callset. The callset (VCF files) then may be filtered using various call metrics to create a final set of high-confidence (such as about or more than about 80%, 85%, 90%, 95%, 99%, or higher confidence) variant calls per sample. Finally, various metrics me be computed per sample, lane, and batch and the calls and metrics are loaded into a laboratory information management system (HMS) for visualization, review, and final report generation. The pipeline can be run (in whole or in part) locally and/or using cloud computing, such as on the Amazon cloud. Users may interact with the pipeline using any suitable communication mechanism. For example, interaction may be via Django management commands (Django Software Foundation, Lawrence, Kans.), a shell script for executing each step of the pipeline, or an application programming interface written in a suitable programming language (e.g. PHP, Ruby on Rails, Django, or an interface like Amazon EC2). Overviews of the operation of this example pipeline are illustrated in FIGS. 10 and 11 of U.S. Pat. No. 9,092,401, which is hereby incorporated by reference in its entirety.

In some embodiments, an alignment according to the invention is performed using a computer program. One exemplary alignment program, which implements a BWT approach, is Burrows-Wheeler Aligner (BWA) available from the SourceForge web site maintained by Geeknet (Fairfax, Va.). The quality of alignments may be assessed and/or compared by calculating an alignment score. For example, the quality of alignments may be assessed and/or compared by calculating an alignment score as described in Heng Li (2013) “Aligning sequence reads, clone sequences and assembly contigs with BWA-MEM” (arXiv:1303.3997v2 [q-bio.GN]). An alignment score for each read or pairs of reads may be used to identify a single top alignment or multiple top alignments for a collection of single-end or paired-end reads. In some cases, the aligner only emits alignments that meet a minimum alignment score for a region of interest, e.g., first, second, or more regions of interest.

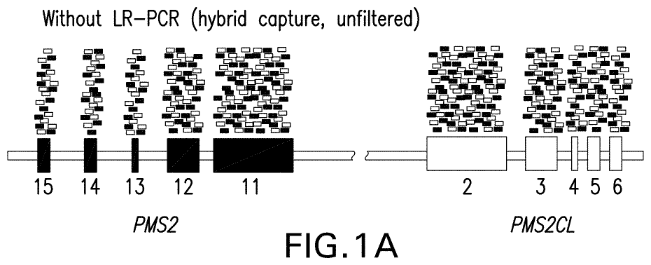

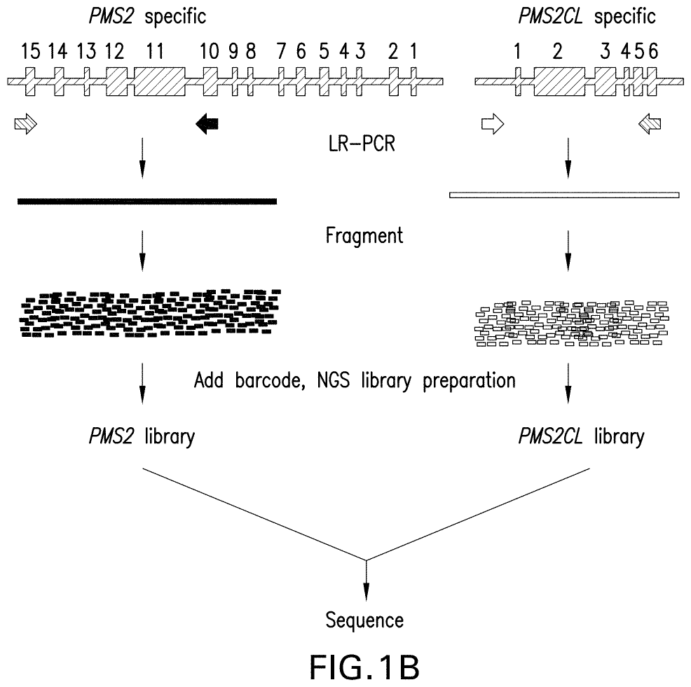

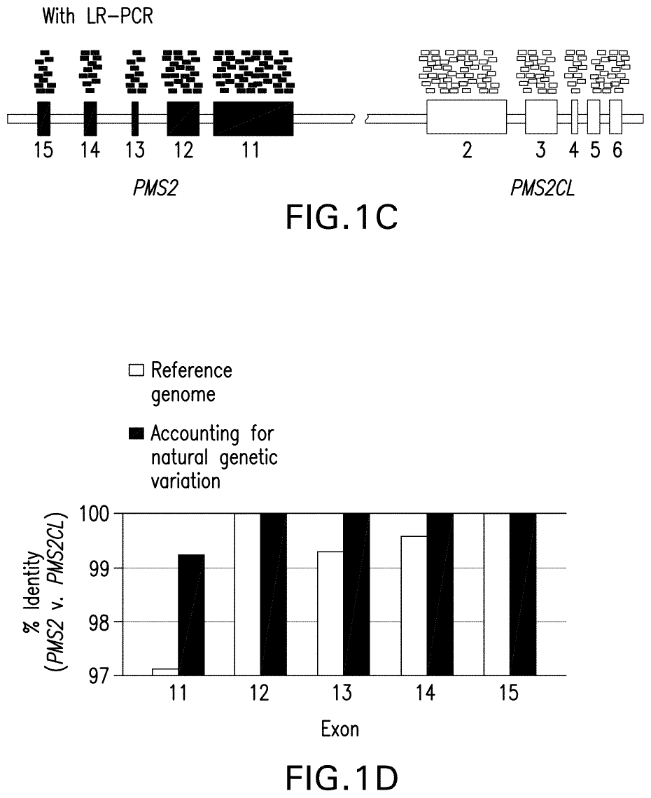

Provided herein are methods for detecting genetic variation in a genome of a subject, wherein the genome comprises highly homologous regions of interest and the genetic variation detected is in one or more of the highly homologous regions of interest. In some embodiments, the highly homologous regions have a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or greater than 99%. In some cases, the method is effective to detect genetic variation between two or more highly homologous regions in the genome. The highly homologous regions may comprise any two or more regions that are highly similar. The homologous regions may comprise two or more genes that are highly similar. In some cases, the homologous regions may comprise one or more gene and one or more homolog of the gene. For example, the homolog may comprise one or more pseudogene. Genotyping such highly homologous regions with standard targeted-NGS strategies that use hybridization to capture and sequence short DNA fragments within each highly homologous region is complicated by the fact that, due to the relatively short read lengths and high homology between the regions, sequence reads cannot be unambiguously aligned to a specific region. For example, PMS2 is commonly included on HCS panels due to its association with Lynch syndrome [11-15]. Its nearby pseudogene, PMS2CL, complicates accurate NGS read alignment and variant identification in exons 11 through 15 at the 3′ end of PMS2 (FIG. 1A): the coding sequences were previously reported to share 98% sequence identity with PMS2CL [16]. Further, sequence exchange and gene conversion between the two regions are sufficiently frequent that even the few non-identical bases in the reference genome (hg19) cannot be reliably attributed to the gene or pseudogene [17,18]. Long-range PCR (LR-PCR) using a gene-specific primer in exon 10 amplifies PMS2 specifically (FIG. 1B), and variants in the terminal five exons of PMS2 can then be identified via Sanger sequencing [19-21] or NGS [22] (FIG. 1C). Although identification of copy-number variants (CNVs) in PMS2 is possible from LR-PCR and Sanger sequencing, it is not straightforward, which has motivated parallel use of multiplex ligation-dependent probe amplification (MLPA) to detect large deletions and duplications [19-24].

Multiple testing strategies exist that can achieve high sensitivity and specificity for highly homologous regions in a genome, for example, PMS2 ([18-20,22,25,26]), although each requires quality-control monitoring. For example, for the last five exons of PMS2, LR-PCR, MLPA, and hybrid-capture NGS on each screened sample was presented previously on a small cohort [22], but applying this combination to a large patient population would be resource intensive and complicate workflow logistics. Herman et al. [26] recently presented a method for identifying CNVs (but not SNVs or indels) in the terminal exons of PMS2 or PMS2CL [26]. The method identified samples for follow-up LR-PCR testing to definitively localize the CNV to the gene or pseudogene. The authors noted a CNV false positive rate of 6.8%, meaning that a significant portion of CNV-negative samples would unnecessarily undergo follow-up testing.

A high reflex rate after short-read NGS testing (e.g., >10%), while acceptable for the accuracy of a patient's report, may exert unmanageable logistical overhead on the testing laboratory. The reflex rate has two components—one biological and one technical—each with different sources and constraints. The biological component serves as the floor of the reflex rate: if the assay had perfect analytical specificity (i.e., zero false positives) and clinical accuracy (i.e., correct classifications with no VUSs), then there would nevertheless be a nonzero reflex rate due to the presence of pathogenic variants in PMS2 exons 12-15 and the corresponding PMS2CL regions that need disambiguation. This biological component would, therefore, reflect primarily the integrated population frequency of pathogenic variants across the ambiguous region. The technical component of the reflex rate, by contrast, arises from imperfect analytical specificity and incomplete knowledge of variant pathogenicity. Though higher in Example 1 (99.7%), analytical specificity for CNVs was 93.7% in Herman et al. [26], meaning that the technical component of the reflex rate in that study was at least 6.3% (highlighting the variable nature of the technical component). Also, technical reflex due to VUSs in the workflow described herein was required in 4% of samples, a share that is expected to drop with further screening of PMS2 and the resulting ability to reclassify VUSs.

Accordingly, a reflex method for detection of variation between homologous regions in a genome is disclosed herein. The method's aim is to have the workflow's initial testing phase (i.e., upstream of reflex) be sensitive enough to maximize detection of PMS2 variants and sufficiently specific to minimize reflex burden. In one embodiment, the method applies hybrid-capture NGS to all samples and LR-PCR/MLPA only as a reflex assay. In some embodiments, the workflow described herein has high analytical accuracy (i.e., is capable of detecting sequence variants in a specific region) while requiring reflex testing for only 10%, 9%, 8%, 7%, 6%, 5%, 4%, 3%, 2%, 1%, or less than 1% of samples. In one embodiment, the workflow described herein has high analytical accuracy while requiring reflex testing for only about 8% of samples. An exemplary embodiment of a method for detection of SNVs, indels, and CNVs in the last five exons of PMS2 is described in Example 1.

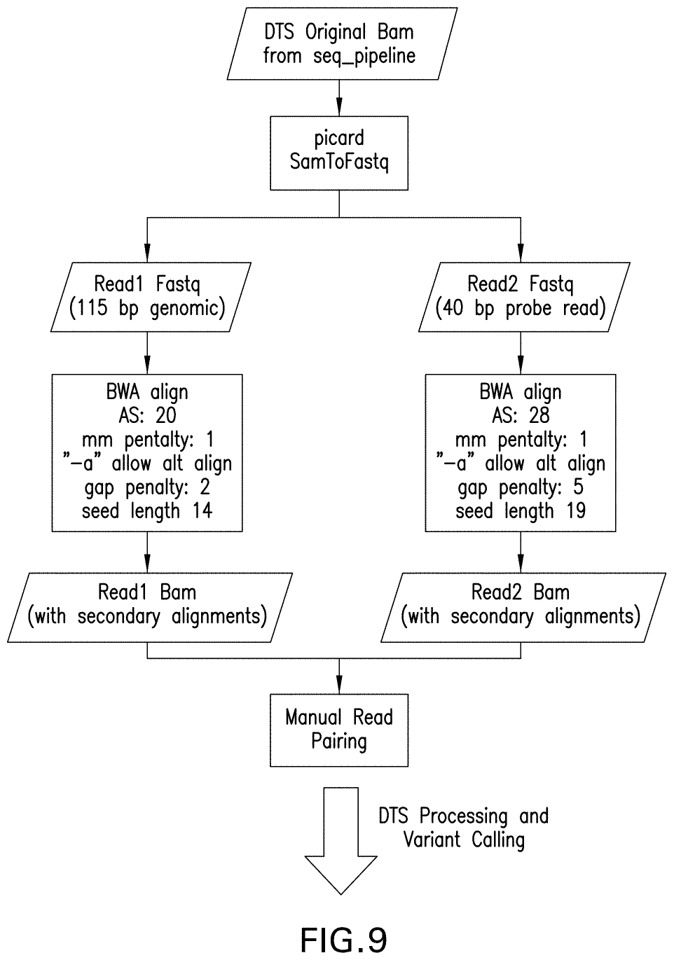

In one embodiment, the method for detecting genetic variation in a genome of a subject, wherein the genome comprises first and second highly homologous regions of interest, comprises: (a) obtaining sequence reads by paired-end sequencing from multiple sites of interest in the first and second regions of interest, wherein the sequence reads comprise a first read and a second read obtained at each site of interest; (b) aligning sequence reads to a reference genome, wherein first reads and second reads are aligned to the reference genome separately and the aligner emits multiple possible alignments for each of the first and second reads; (c) identifying first reads and second reads that align to the first region of interest; (d) pairing a first read and a second read from the reads identified in step (c), thereby generating a top paired alignment; and (e) detecting the genetic variation in the top paired alignment generated in step (d). In a preferred embodiment, reads are aligned to a reference genome, wherein the reference genome does not comprise a masked or modified portion of a first or second homologous region of interest, wherein the first and/or second homologous regions of interest is/are being analyzed to detect genetic variation as described herein. The alignment in step (b) is referred to as an “ambiguous alignment”, because each single-end sequence read is separately aligned to the reference genome and multiple read alignments are identified in (c). An example of this method's implementation via the “ambiguous alignment” process is diagrammed in FIG. 9.

In another embodiment, the method for detecting genetic variation in a genome of a subject, wherein the genome comprises first and second highly homologous regions of interest, comprises: (a) obtaining sequence reads by paired-end sequencing from multiple sites of interest in the first and second regions of interest, wherein the sequence reads comprise a first read and a second read obtained at each site of interest; (b) aligning first reads and second reads to a reference genome, wherein the aligner emits the best possible paired-end alignment to the first or second region of interest for each pair of first and second reads, and wherein only paired-end reads associated with a top alignment score to the first or second regions of interest are aligned separately in step (c); (c) aligning sequence reads to a reference genome, wherein first reads and second reads are aligned to the reference genome separately and the aligner emits multiple possible alignments for each of the first and second reads; (d) identifying first reads and second reads that align to the first region of interest; (e) pairing a first read and a second read from the reads identified in step (d), thereby generating a top paired alignment; and (f) detecting the genetic variation in the top paired alignment generated in step (e). In a preferred embodiment, reads are aligned to a reference genome, wherein the reference genome does not comprise a masked or modified portion of a first or second homologous region of interest, wherein the first and/or second homologous regions of interest is/are being analyzed to detect genetic variation as described herein. Thus, in some embodiments, a standard paired-end alignment is performed initially to select for reads that align to a region of interest, wherein typically only paired-end reads with the top alignment score are selected. Next, the selected paired-end reads may be partitioned and separately aligned to the reference genome to identify multiple top single-end alignments for each read (e.g., “ambiguous alignment”).

The multiple top single-end alignments emitted by the aligner for each read may be individually paired to generate a top paired alignment. For example, top paired-end reads are partitioned into a BAM file, for example using samtools [28], the BAM file is converted into two unaligned FASTQ files (each member of the read pair parsed to one of the two files), for example using Picard (Broad Institute), and each single-end FASTQ file is separately realigned to a reference genome allowing for “ambiguous alignment” and reporting of the top several alignments for each read. Such top alignments may be used in the pairing step, to identity a top paired alignment.

Single-end reads selected through “ambiguous alignment” may be used to generate a top paired-end alignment through a selection process. Single-end alignments may be used to generate a top paired-end alignment if: 1) both single end reads have the same read name; 2) both single-end reads map to the region spanning the region of interest used to identify single-end reads via “ambiguous alignment” as described above; and/or 3) both single-end reads align within a certain number of bases of each other. In a preferred embodiment, only reads that meet all of pairing criteria (1)-(3) are paired. In some embodiments, reads are paired only if the alignments of the first read and the second read in the region of interest used to identify single-end reads via “ambiguous alignment” as described above are within about 100 bp, about 200 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp, about 1500 bp, or more than 1500 bp. In some cases, when multiple putative pairs meet the above conditions for a given read name, the pair with the highest alignment score is chosen. In some cases, a top paired-end alignment is selected as having the smallest template length. Reads that cannot form proper pairs as described above are discarded. The resulting paired-end BAM file contains reads originating from both homologous regions of interest, mapped to the region of interest used to identify single-end reads via “ambiguous alignment”. The top paired-end alignment can be analyzed to identify or call variants in the one or more homologous regions of interest.

For example, for PMS2, resulting single-end alignments may be used to generate a paired-end alignment if the following criteria are met: 1) both single end reads have the same read name; 2) both single-end reads map to the region spanning PMS2 exons 12-15; 3) both single-end reads align within 1000 bp of each other; 4) when multiple putative pairs met the above conditions for a given read name, the pair with the highest alignment score is chosen, and 5) reads that cannot form proper pairs as described above are discarded. The resulting paired-end BAM file contains reads originating from both PMS2 and PMS2CL reads, mapped to the PMS2 sequence.

In one embodiment, the genetic variation detected in the homologous sequences comprises one of more SNPs. In another embodiment, the genetic variation detected in the homologous sequences comprises one of more CNVs. In another embodiment, the genetic variation detected in the homologous sequences comprises one of more indels. In another embodiment, the genetic variation detected in the homologous sequences comprises one of more inversions. In another embodiment, the genetic variation detected in the homologous sequences comprises a combination of SNPs, indels, inversions, and/or CNVs.

In one embodiment, to detect genetic variation in a genome of a subject as described herein, wherein the genome comprises highly homologous regions comprising a first and a second region of interest, sequence reads are obtained from one or more exons within the first and/or second region(s) of interest. Sequence reads may be obtained from one or more introns within the first and/or second region(s) of interest. Sequence reads may be obtained from one or more exons and introns within the first and/or second region(s) of interest. Sequence reads may be obtained from one or more exons and introns within the first and/or second region(s) of interest, wherein the introns are near the exons. An intron that is near an exon may be within +/−1-100 nt, for example +/−20 nt, of the exon. Sequence reads may be obtained from one or more clinically actionable regions associated with the first and/or second region(s) of interest. Such regions associated with the first and/or second region(s) of interest may include any region of the genome. For example, the clinically actionable regions may include a promoter, an enhancer, and/or an untranslated region. In some cases, the first region of interest comprises a gene and the second region of interest comprises a pseudogene. In other cases, the first region of interest may comprise a pseudogene and the second region of interest comprises a gene. The first region of interest may comprise two alleles. The second region of interest may comprise two alleles.

In one embodiment, if a genetic variation is detected in highly homologous regions of interest in a subject's genome according to the methods described herein, a portion of the subject's genome is amplified by long-range PCR and assayed by multiplex ligation-dependent probe amplification (MLPA). In another embodiment, if a genetic variation is detected in highly homologous regions of interest in a subject's genome according to the methods described herein, a portion of the first region of interest is amplified by long-range PCR and the product or a portion thereof is sequenced by Sanger sequencing. In another embodiment, if a genetic variation is detected in highly homologous regions of interest in a subject's genome according to the methods described herein, a portion of the first region of interest is amplified by long-range PCR and the product or a portion thereof is sequenced by NGS. In another embodiment, if a genetic variation is detected in highly homologous regions of interest in a subject's genome according to the methods described herein, the subject's genomic DNA is assayed by multiplex ligation-dependent probe amplification (MLPA).

In an embodiment, the gene is PMS2 and the pseudogene is either PMS2CL or one of several other pseudogenes for PMS2. The pseudogenes for exons 9 and 11-15 of PMS2 may be selected from, but not limited to, PMS2CL. The pseudogenes for all of PMS2, but especially exons 1-5 of PMS2, may be selected from, but not limited to, 15 or more/fewer pseudogenes. In an embodiment, the presence of an altered copy number and/or inversions that alter orientation of the gene and pseudogene (e.g., those that fuse portions of pseudogene with the gene and thus compromise gene function) may indicate that the subject has increased risk for the disease Lynch Syndrome.

In one embodiment, the multiple sites of interest in the highly homologous regions from which the paired-end reads are obtained are within an exon of PMS2 and an exon in another part of the subject's genome. In another embodiment, the multiple sites of interest are within an exon of PMS2 and an exon of PMS2CL. In another embodiment, the multiple sites of interest are within exons 11, 12, 13, 14, and/or 15 of PMS2 and exons 2, 3, 4, 5, and/or 6 of PMS2CL.

In one embodiment, the gene is SMN1 and the pseudogene is SMN2. In an embodiment, the presence of an altered copy number of SMN1 indicates that the subject may be a carrier for the disease spinal muscular atrophy (SMA).

In another embodiment, the gene is CYP21A2 and the pseudogene is CYP21A1P. In an embodiment, the presence of an altered copy number of CYP21A2 indicates that the subject may be a carrier for the disease congenital adrenal hyperplasia (CAH).

In an embodiment, the gene is HBA1 and the homolog is HBA2 (or vice versa). In an embodiment, the presence of an altered copy number of either HBA1 or HBA2 indicates that the subject may be a carrier for the disease alpha-thalassemia.

In a further embodiment, the gene is GBA and the pseudogene is GBAP. In an embodiment, the presence of an altered copy number of GBA indicates that the subject may be a carrier for the disease Gaucher's Disease.

In an embodiment, the gene is CHEK2, which has several pseudogenes. As of December 2014, there were seven pseudogenes. The pseudogenes may be selected from, but not limited to, CHEK2 pseudogenes enumerated in a curated database. In an embodiment, the presence of mutations that arise from recombination with its pseudogenes—e.g., a pseudogene-derived frameshift mutation—may indicate that the subject has increased risk for the disease breast cancer, among other diseases. It is well known in the art that only one of the seven pseudogenes has been named and that risk is primarily associated with one mutation, 1100delC. However, other mutations also contribute to risk of disease. Patients are at risk for Li Fraumeni syndrome and other heritable cancers.

In an embodiment, the gene is SDHA, and the pseudogene is any one of its pseudogenes, for example, SDHAP1, SDHAP2, SDHAP3.

### III. Variant Calling

In some embodiments, variants are detected with a computer-implemented caller algorithm. In principle, any variant caller may be utilized, e.g., to detect SNPs, indels, inversions, and CNVs. In some cases, a caller is used that is capable of detecting/resolving breakpoints when genetic variation, e.g., a deletion, is detected. For example, a caller may be selected from a caller cited in Tattini, L., et al., Front Bioeng Biotechnol. 2015; 3: 92. In some cases, variants are identified based on an expected ploidy of 0-7, or 0-8. In some cases, variants are identified based on an expected ploidy of 2. In other cases, variants are identified based on an expected ploidy of 6. In other cases, variants are identified based on an expected ploidy of 4. For example, SNVs and indels may be identified using GATK 4.0 HaplotypeCaller [29] with the sample-ploidy option set to 4 (e.g., for the tetraploid PMS2 exon 12-15 regions). In other cases, SNVs and short indels may be identified using GATK 1.6 [30] and FreeB ayes [31] with the sample-ploidy option set to 2 (e.g., for the diploid PMS2 exon 11 region). For diploid SNV calling in LR-PCR data, GATK 1.6 may be similarly used.

In a preferred embodiment, a hidden Markov model (HMM) caller is used to determine a copy number. A preferred caller used to determine a copy number is the HMM caller described in U.S. Provisional Patent Application No. 62/681,517, which is hereby incorporated by reference in its entirety. In some embodiments, a preferred HMM caller is set to an expected ploidy of 2. In other embodiments, a preferred HMM caller is set to an expected ploidy of 4. In other embodiments, a preferred HMM caller is set to an expected ploidy of 6.

In some embodiments, methods of assessing a sample-specific performance of a copy number variant model, a method for determining a copy number of an interrogated segment within a region of interest, and a method for determining a copy number variant abnormality within a region of interest are utilized as described in U.S. Provisional patent Application No. 62/681,517, which is hereby incorporated by reference in its entirety.

In some embodiments, a method of assessing the sample-specific performance of a copy number variant caller comprising a copy number variant model is utilized, comprising: parameterizing the copy number variant model based on real numbers of sequencing reads mapped to segments within a region of interest, from a test sample, to determine one or more copy number variant model parameters; generating a plurality of synthetic copy number variants, each synthetic copy number variant comprising a synthetic number of copies of one or more of the segments, wherein each synthetic number of copies is represented by a synthetic number of sequencing reads based on a real number of sequencing reads for a corresponding segment from the test sample; calling a number of copies of the one or more segments for the synthetic copy number variants using the copy number variant model, and the one or more determined copy number variant model parameters; determining a sample-specific performance statistic for the copy number variant caller based on differences between the called number of copies and the synthetic number of copies in the synthetic copy number variants; and assessing a sample-specific performance of the copy number variant caller based on the sample-specific performance statistic.

In some embodiments of the method of assessing the sample-specific performance of the copy number variant caller, the synthetic number of sequencing reads for the one or more segments is generated by increasing, decreasing, or maintaining the real number of sequencing reads for the corresponding segments from the test sample in proportion to a predetermined number of copies of the one or more segments. In some embodiments, the predetermined number of copies is an integer number of copies. In some embodiments, the predetermined number of copies is a non-integer number of copies.

In some embodiments of the method of assessing the sample-specific performance of the copy number variant caller, the synthetic number of sequencing reads is generated by sampling a binomial distribution with a success probability equal to m/x and a number of trials equal to the real number of sequencing reads at the corresponding segment from the test sample, wherein m is the synthetic number of copies of the segment in the synthetic copy number variant, and x is an assumed number of copies of the corresponding segment from the test sample.

In some embodiments of the method of assessing the sample-specific performance of the copy number variant caller, the synthetic number of sequencing reads is generated by: sampling a number of sequencing reads as a negative binomial distribution with a success probability equal to mix and a number of successes equal to the real number of sequencing reads at the corresponding segment from the test sample, wherein m is the synthetic number of copies of the segment in the synthetic copy number variant, and x is an assumed number of copies of the corresponding segment from the test sample, and adding the sampled number of sequencing reads to the real number of sequencing reads for the corresponding segment from the test sample. In some embodiments, the synthetic number of sequencing reads is generated by sampling a number of sequencing reads as an expectation of the negative binomial distribution.

In some embodiments of the method of assessing the sample-specific performance of the copy number variant caller, the copy number variant model is a hidden Markov model. In some embodiments, the hidden Markov model comprises: (i) one or more hidden states comprising a copy number corresponding to an interrogated segment or a plurality of sub-segments within the interrogated segment; (ii) an observation state comprising the real or synthetic number of sequencing reads for the interrogated segment; (iii) a copy number likelihood model based on an expected number of real or synthetic sequencing reads for the interrogated segment. In some embodiments, the method comprises determining the copy number likelihood model. In some embodiments, parameterizing the hidden Markov model comprises adjusting the copy number likelihood model to fit the real number of sequencing reads mapped to the interrogated segment, from the test sample. In some embodiments, the copy number likelihood model comprises a distribution for two or more copy number states. In some embodiments, the copy number likelihood model comprises a negative binomial distribution, wherein the negative binomial distribution is not a Poisson distribution. In some embodiments, the expected number of real or synthetic sequencing reads is based on an average number of mapped sequencing reads at a segment corresponding to the interrogated segment across a plurality of samples, and an average number of mapped sequencing reads across the segments within the test sample, wherein the average number of mapped sequencing reads at the segment corresponding to the interrogated segment across the plurality of samples or the average number of mapped sequencing reads across the plurality of segments within the test sample is a normalized average. In some embodiments, the copy number likelihood model is adjusted to account for the presence of GC content bias. In some embodiments, the hidden Markov model comprises a transition probability of the copy number of the interrogated segment for a given copy number of a spatially adjacent segment. In some embodiments, the hidden Markov model comprises a plurality of transition probabilities of the copy number of a sub-segment in the plurality of sub-segments within the interrogated segment for a given copy number of a spatially adjacent sub-segment. In some embodiments, the transition probability accounts for an average length of a copy number variant. In some embodiments, the transition probability accounts for a prior probability of a copy number variant at the interrogated segment or a spatially adjacent segment. In some embodiments, the average length of a copy number variant or the probability of a copy number variant at the interrogated segment is determined based on observations in a human population.

In some embodiments of the method of assessing the sample-specific performance of the copy number variant caller, parameterizing the copy number variant model comprises accounting for one or more spurious capture probes. In some embodiments, accounting for one or more spurious capture probes comprises weighting the one or more observation states in the plurality of observation states with a spurious capture probe indicator. In some embodiments, the spurious capture probe indicator is determined using a Bernoulli process. In some embodiments, accounting for one or more of the capture probes being spurious comprises using expectation-maximization. In some embodiments, if a capture probe is determined to be spurious, sequencing reads derived from that capture probe is disregarded in the copy number variant model.

In some embodiments of the method of assessing the sample-specific performance of the copy number variant caller, parameterizing of the copy number variant model comprises accounting for noise in the number of mapped sequencing reads.

In some embodiments of the method of assessing the sample-specific performance of the copy number variant caller, the copy number variant model is parameterized using an analytic first derivative gradient and second derivative Hessian of one or more copy number variant model parameters.

In some embodiments of the method of assessing the sample-specific performance of the copy number variant caller, the copy number variant model is parameterized by solving a trust region Newton conjugate gradient algorithm.

In some embodiments of the method of assessing the sample-specific performance of the copy number variant caller, the copy number variant model is iteratively parameterized using expectation-maximization.

In some embodiments of the method of assessing the sample-specific performance of the copy number variant caller, the method comprises mapping the real sequencing reads from the test sample to the segments within the region of interest, and determining the real numbers of sequencing reads mapped to the segments.

In some embodiments of the method of assessing the sample-specific performance of the copy number variant caller, the test sample is enriched using one or more direct targeted sequencing capture probes.

In some embodiments of the method of assessing the sample-specific performance of the copy number variant caller, the method comprises calling a copy number of the one or more segments for the test sample.

In some embodiments of the method of assessing the sample-specific performance of the copy number variant caller, the segments comprise spatially adjacent segments.

In some embodiments of the method of assessing the sample-specific performance of the copy number variant caller, the sample-specific performance statistic is a limit of detection, sensitivity, specificity, precision, recall, accuracy, positive predictive value, or negative predictive value.

In some embodiments of the method of assessing the sample-specific performance of the copy number variant caller, the sample-specific performance statistic is sensitivity or accuracy.

In some embodiments of the method of assessing the sample-specific performance of the copy number variant caller, the method comprises failing the test sample if the sample-specific performance of the copy number variant model is below a desired performance threshold.

Also described herein is a method for determining a copy number of an interrogated segment within a region of interest comprising: (a) mapping a plurality of sequencing reads generated from a test sequencing library to the interrogated segment, wherein the test sequencing library is enriched using one or more direct targeted sequencing capture probes; (b) determining a number of sequencing reads mapped to the interrogated segment; (c) determining a copy number likelihood model based on an expected number of sequencing reads mapped to the interrogated segment; (d) building a hidden Markov model comprising: (i) one or more hidden states comprising a copy number corresponding to the interrogated segment or a plurality of sub-segments within the interrogated segment, (ii) an observation state comprising the number of sequencing reads mapped to the interrogated segment; and (iii) the copy number likelihood model; (e) parameterizing the hidden Markov model by adjusting the copy number likelihood model to fit the determined number of sequencing reads mapped to the interrogated segment, wherein the hidden Markov model is parameterized using an analytic first derivative gradient and second derivative Hessian of one or more parameters in the copy number likelihood model; and (f) determining a most probable copy number of the interrogated segment based on the parameterized hidden Markov model.

Further described herein is a method for determining a copy number of an interrogated segment within a region of interest comprising: (a) mapping a plurality of sequencing reads generated from a test sequencing library to a plurality of spatially adjacent segments, wherein the plurality of spatially adjacent segments comprises the interrogated segment, and wherein the test sequencing library is enriched using a plurality of spatially adjacent direct targeted sequencing capture probes; (b) determining a number of sequencing reads mapped to each spatially adjacent segment; (c) determining a copy number likelihood model for each spatially adjacent segment based on an expected number of mapped sequencing reads at the spatially adjacent segment; (d) building a hidden Markov model comprising: (i) a plurality of hidden states comprising a copy number for each of the spatially adjacent segments or a plurality of sub-segments within each of the spatially adjacent segments, (ii) a plurality of observation states comprising the number of sequencing reads mapped to each spatially adjacent segment, and (iii) the copy number likelihood model for each spatially adjacent segment; (e) parameterizing the hidden Markov model, comprising adjusting each copy number likelihood model to fit the determined number of sequencing reads mapped to each spatially adjacent segment, wherein the hidden Markov model is parameterized using an analytic first derivative gradient and second derivative Hessian of one or more parameters in the copy number likelihood model; and (f) determining a most probable copy number of the interrogated segment based on the parameterized hidden Markov model.

Also described herein is a method for determining a copy number variant abnormality within a region of interest, comprising: (a) mapping a plurality of sequencing reads generated from a test sequencing library to an interrogated segment within the region of interest, wherein the test sequencing library is enriched using one or more direct targeted sequencing capture probes; (b) determining a number of sequencing reads mapped to the interrogated segment; (c) determining a copy number likelihood model based on an expected number of sequencing reads mapped to the interrogated segment; (d) building a hidden Markov model comprising: (i) one or more hidden states comprising a copy number corresponding to the interrogated segment or a plurality of sub-segments within the interrogated segment, (ii) an observation state comprising the number of sequencing reads mapped to the interrogated segment; and (iii) the copy number likelihood model; (e) parameterizing the hidden Markov model by adjusting the copy number likelihood model to fit the determined number of sequencing reads mapped to the interrogated segment, wherein the hidden Markov model is parameterized using an analytic first derivative gradient and second derivative Hessian of one or more parameters in the copy number likelihood model; and (f) determining a most probable copy number of the interrogated segment based on the parameterized hidden Markov model; (g) determining a copy number variant abnormality based on the most probable copy number of the interrogated segment.

Further described herein is a method for determining a copy number variant abnormality within a region of interest, comprising: (a) mapping a plurality of sequencing reads generated from a test sequencing library to a plurality of spatially adjacent segments, wherein the plurality of spatially adjacent segments comprises an interrogated segment, and wherein the test sequencing library is enriched using a plurality of spatially adjacent direct targeted sequencing capture probes; (b) determining a number of sequencing reads mapped to each spatially adjacent segment; (c) determining a copy number likelihood model for each spatially adjacent segment based on an expected number of mapped sequencing reads at the spatially adjacent segment; (d) building a hidden Markov model comprising: (i) a plurality of hidden states comprising a copy number for each of the spatially adjacent segments or a plurality of sub-segments within each of the spatially adjacent segments, (ii) a plurality of observation states comprising the number of sequencing reads mapped to each spatially adjacent segment, and (iii) the copy number likelihood model for each spatially adjacent segment; (e) parameterizing the hidden Markov model comprising adjusting each copy number likelihood model to fit the determined number of sequencing reads mapped to each spatially adjacent segment, wherein the hidden Markov model is parameterized using an analytic first derivative gradient and second derivative Hessian of one or more parameters in the copy number likelihood model; and (f) determining a most probable copy number of the interrogated segment based on the parameterized hidden Markov model; (g) determining a copy number variant abnormality based on the most probable copy number of the interrogated segment.

Also described herein is a method for determining a copy number of an interrogated segment within a region of interest comprising: (a) mapping a plurality of sequencing reads generated from a test sequencing library to the interrogated segment, wherein the test sequencing library is enriched using one or more capture probes; (b) determining a number of sequencing reads mapped to the interrogated segment; (c) determining a copy number likelihood model based on an expected number of sequencing reads mapped to the interrogated segment; (d) building a hidden Markov model comprising: (i) one or more hidden states comprising a copy number corresponding to the interrogated segment or a plurality of sub-segments within the interrogated segment, (ii) an observation state comprising the number of sequencing reads mapped to the interrogated segment; and (iii) the copy number likelihood model; (e) parameterizing the hidden Markov model by adjusting the copy number likelihood model to fit the determined number of sequencing reads mapped to the interrogated segment and accounting for one or more spurious capture probes, wherein the hidden Markov model is parameterized using an analytic first derivative gradient and second derivative Hessian of one or more parameters in the copy number likelihood model; and (f) determining a most probable copy number of the interrogated segment based on the parameterized hidden Markov model.

Further described herein is a method for determining a copy number of an interrogated segment within a region of interest comprising: (a) mapping a plurality of sequencing reads generated from a test sequencing library to a plurality of spatially adjacent segments, wherein the plurality of spatially adjacent segments comprises the interrogated segment, and wherein the test sequencing library is enriched using a plurality of spatially adjacent direct targeted sequencing capture probes; (b) determining a number of sequencing reads mapped to each spatially adjacent segment; (c) determining a copy number likelihood model for each spatially adjacent segment based on an expected number of mapped sequencing reads at the spatially adjacent segment; (d) building a hidden Markov model comprising: (i) a plurality of hidden states comprising a copy number for each of the spatially adjacent segments or a plurality of sub-segments within each of the spatially adjacent segments, (ii) a plurality of observation states comprising the number of sequencing reads mapped to each spatially adjacent segment, and (iii) the copy number likelihood model for each spatially adjacent segment; (e) parameterizing the hidden Markov model comprising adjusting each copy number likelihood model to fit the determined number of sequencing reads mapped to each spatially adjacent segment and accounting for one or more spurious capture probes, wherein the hidden Markov model is parameterized using an analytic first derivative gradient and second derivative Hessian of one or more parameters in the copy number likelihood model; and (f) determining a most probable copy number of the interrogated segment based on the parameterized hidden Markov model.

In some embodiments of the methods described above, the one or more parameters of the copy number likelihood model comprises a dispersion of a number of mapped sequencing reads for the segment (dᵢ), an average number of mapped sequencing reads for the segment (μᵢ), a dispersion of a number of mapped sequencing reads for the segments within the test sequencing library (dⱼ), or an average number of mapped sequencing reads for the segments within the test sequencing library (μⱼ).

In some embodiments of the methods described above, the method further comprises determining a most probable copy number of a section within the region of interest, wherein the section comprises a plurality of spatially adjacent segments comprising the interrogated segment.

In some embodiments of the methods described above, the copy number likelihood model comprises a distribution for two or more copy number states.

In some embodiments of the methods described above, the copy number likelihood model comprises a negative binomial distribution, wherein the negative binomial distribution is not a Poisson distribution.

In some embodiments of the methods described above, the expected number of sequencing reads is based on an average number of mapped sequencing reads at a corresponding segment across a plurality of sequencing libraries and an average number of mapped sequencing reads across a plurality of segments of interest within the test sequencing library, wherein the average number of mapped sequencing reads at a corresponding segment across a plurality of sequencing libraries or the average number of mapped sequencing reads across a plurality of segments of interest within the test sequencing library is a normalized average.

In some embodiments of the methods described above, the copy number likelihood model is adjusted to account for the presence of GC content bias. In some embodiments, the adjustment depends on the GC content of the capture probe corresponding to the interrogated segment or the GC content of the interrogated segment.

In some embodiments of the methods described above, the hidden Markov model comprises a transition probability of the copy number of the interrogated segment for a given copy number of a spatially adjacent segment. In some embodiments, the transition probability accounts for an average length of a copy number variant. In some embodiments, the transition probability accounts for a prior probability of a copy number variant at the interrogated segment or a spatially adjacent segment. In some embodiments, the average length of a copy number variant or the probability of a copy number variant at the interrogated segment are determined based on observations in a human population.

In some embodiments of the methods described above, the hidden Markov model comprises a plurality of transition probabilities of the copy number of a sub-segment in the plurality of sub-segments within the interrogated segment for a given copy number of a spatially adjacent sub-segment. In some embodiments, the transition probability accounts for an average length of a copy number variant. In some embodiments, the transition probability accounts for a prior probability of a copy number variant at the interrogated segment or a spatially adjacent segment. In some embodiments, the average length of a copy number variant or the probability of a copy number variant at the interrogated segment are determined based on observations in a human population.

In some embodiments of the methods described above, parameterizing the hidden Markov model comprises accounting for one or more spurious capture probes. In some embodiments, accounting for one or more spurious capture probes comprises weighting the one or more observation states in the plurality of observation states with a spurious capture probe indicator. In some embodiments, the spurious capture probe indicator is determined using a Bernoulli process. In some embodiments, accounting for one or more of the capture probes being spurious comprises using expectation-maximization. In some embodiments, if a capture probe is determined to be spurious, the likelihood information from that capture probe is disregarded in the copy number likelihood model.

In some embodiments of the methods described above, parameterizing of the hidden Markov model comprises accounting for noise in the number of mapped sequencing reads.

In some embodiments of the methods described above, accounting for noise in the number of mapped sequencing reads comprises adjusting the copy number likelihood model. In some embodiments adjusting the copy number likelihood model to account for the noise comprises an expectation-maximization step. In some embodiments, the expectation-maximization step comprises weighing a level of noise in the number of mapped sequencing reads from the test sequencing library. In some embodiments, the most probable copy number of the interrogated segment is not called if the noise in the number of mapped sequencing reads is above a predetermined threshold.

In some embodiments of the methods described above, sequencing reads from overlapping capture probes are merged.

In some embodiments of the methods described above, a Viterbi algorithm, a Quasi-Newton solver, or a Markov chain Monte Carlo is used to determine the most probable copy number of the interrogated segment.

In some embodiments of the methods described above, the method further comprises determining a confidence of the most probable copy number of the segment.

In some embodiments of the methods described above, the one or more parameters of the copy number likelihood model comprises a dispersion of a number of mapped sequencing reads for the segment (dᵢ), an average number of mapped sequencing reads for the segment (μᵢ), a dispersion of a number of mapped sequencing reads for the segments within the test sequencing library (dⱼ), or an average number of mapped sequencing reads for the segments within the test sequencing library (μⱼ).

In some embodiments of the methods described above, the analytic first derivative gradient and second derivative analytical Hessian of the one or more parameters in the copy number likelihood model is solved using a trust region Newton conjugate gradient algorithm.

Also described herein is a computer system comprising a computer-readable medium comprising instructions for carrying out any one of the methods described above.

### IV. Exemplary Architecture and Processing Environment

In a preferred embodiment, a portion of the methods described herein are computer-implemented. An exemplary environment and system in which certain aspects and examples of the systems and processes described herein may operate. As shown in FIG. 10, in some examples, the system can be implemented according to a client-server model. The system can include a client-side portion executed on a user device 102 and a server-side portion executed on a server system 110. User device 102 can include any electronic device, such as a desktop computer, laptop computer, tablet computer, PDA, mobile phone (e.g., smartphone), or the like.

User devices 102 can communicate with server system 110 through one or more networks 108, which can include the Internet, an intranet, or any other wired or wireless public or private network. The client-side portion of the exemplary system on user device 102 can provide client-side functionalities, such as user-facing input and output processing and communications with server system 110. Server system 110 can provide server-side functionalities for any number of clients residing on a respective user device 102. Further, server system 110 can include one or caller servers 114 that can include a client-facing I/O interface 122, one or more processing modules 118, data and model storage 120, and an I/O interface to external services 116. The client-facing I/O interface 122 can facilitate the client-facing input and output processing for caller servers 114. The one or more processing modules 118 can include various issue and candidate scoring models as described herein. In some examples, caller server 114 can communicate with external services 124, such as text databases, subscriptions services, government record services, and the like, through network(s) 108 for task completion or information acquisition. The I/O interface to external services 116 can facilitate such communications.

Server system 110 can be implemented on one or more standalone data processing devices or a distributed network of computers. In some examples, server system 110 can employ various virtual devices and/or services of third-party service providers (e.g., third-party cloud service providers) to provide the underlying computing resources and/or infrastructure resources of server system 110.

Although the functionality of the caller server 114 is shown in FIG. 10 as including both a client-side portion and a server-side portion, in some examples, certain functions described herein (e.g., with respect to user interface features and graphical elements) can be implemented as a standalone application installed on a user device. In addition, the division of functionalities between the client and server portions of the system can vary in different examples. For instance, in some examples, the client executed on user device 102 can be a thin client that provides only user-facing input and output processing functions, and delegates all other functionalities of the system to a backend server.

It should be noted that server system 110 and clients 102 may further include any one of various types of computer devices, having, e.g., a processing unit, a memory (which may include logic or software for carrying out some or all of the functions described herein), and a communication interface, as well as other conventional computer components (e.g., input device, such as a keyboard/touch screen, and output device, such as display). Further, one or both of server system 110 and clients 102 generally includes logic (e.g., http web server logic) or is programmed to format data, accessed from local or remote databases or other sources of data and content. To this end, server system 110 may utilize various web data interface techniques such as Common Gateway Interface (CGI) protocol and associated applications (or “scripts”), Java® “servlets,” i.e., Java® applications running on server system 110, or the like to present information and receive input from clients 102. Server system 110, although described herein in the singular, may actually comprise plural computers, devices, databases, associated backend devices, and the like, communicating (wired and/or wireless) and cooperating to perform some or all of the functions described herein. Server system 110 may further include or communicate with account servers (e.g., email servers), mobile servers, media servers, and the like.

It should further be noted that although the exemplary methods and systems described herein describe use of a separate server and database systems for performing various functions, other embodiments could be implemented by storing the software or programming that operates to cause the described functions on a single device or any combination of multiple devices as a matter of design choice so long as the functionality described is performed. Similarly, the database system described can be implemented as a single database, a distributed database, a collection of distributed databases, a database with redundant online or offline backups or other redundancies, or the like, and can include a distributed database or storage network and associated processing intelligence. Although not depicted in the figures, server system 110 (and other servers and services described herein) generally include such art recognized components as are ordinarily found in server systems, including but not limited to processors, RAM, ROM, clocks, hardware drivers, associated storage, and the like (see, e.g., FIG. 11, discussed below). Further, the described functions and logic may be included in software, hardware, firmware, or combination thereof.

FIG. 11 depicts an exemplary computing system 1400 configured to perform any one of the above-described processes, including the various calling and scoring models. In this context, computing system 1400 may include, for example, a processor, memory, storage, and input/output devices (e.g., monitor, keyboard, disk drive, Internet connection, etc.). However, computing system 1400 may include circuitry or other specialized hardware for carrying out some or all aspects of the processes. In some operational settings, computing system 1400 may be configured as a system that includes one or more units, each of which is configured to carry out some aspects of the processes either in software, hardware, or some combination thereof.

FIG. 11 depicts computing system 1400 with a number of components that may be used to perform the above-described processes. The main system 1402 includes a motherboard 1404 having an input/output (“I/O”) section 1406, one or more central processing units (“CPU”) 1408, and a memory section 1410, which may have a flash memory card 1412 related to it. The I/O section 1406 is connected to a display 1424, a keyboard 1414, a disk storage unit 1416, and a media drive unit 1418. The media drive unit 1418 can read/write a computer-readable medium 1420, which can contain programs 1422 and/or data.

At least some values based on the results of the above-described processes can be saved for subsequent use. Additionally, a non-transitory computer-readable medium can be used to store (e.g., tangibly embody) one or more computer programs for performing any one of the above-described processes by means of a computer. The computer program may be written, for example, in a general-purpose programming language (e.g., Pascal, C, C++, Python, Java) or some specialized application-specific language.

Various exemplary embodiments are described herein. Reference is made to these examples in a non-limiting sense. They are provided to illustrate more broadly applicable aspects of the disclosed technology. Various changes may be made and equivalents may be substituted without departing from the true spirit and scope of the various embodiments. In addition, many modifications may be made to adapt a particular situation, material, composition of matter, process, process act(s) or step(s) to the objective(s), spirit or scope of the various embodiments. Further, as will be appreciated by those with skill in the art, each of the individual variations described and illustrated herein has discrete components and features that may be readily separated from or combined with the features of any of the other several embodiments without departing from the scope or spirit of the various embodiments. All such modifications are intended to be within the scope of claims associated with this disclosure.

### EXAMPLES

The present invention is described in further detain in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

### Example 1

### Detecting Clinically Actionable Variants in the 3′ Exons of PMS2

This example illustrates a strategy for detection of SNVs, indels, and CNVs in the 3′ exons of PMS2. This study was reviewed and designated as exempt by Western Institutional Review Board and complied with the Health Insurance Portability and Accountability Act (HIPAA).

### Materials and Methods

### Study Samples

Table S1 of Appendix indicates which sample sets were used for particular assays and analyses. Cell-line DNA was purchased from Coriell Cell Repositories (Camden, N.J.) (Table S2 of Appendix). Patient sample DNA was extracted from de-identified blood or saliva samples. DNA samples with known positives were a gift from Invitae Corporation.

### LR-PCR

DNA was extracted and underwent an additional cleanup via incubation with 1×SPRI beads followed by 80% ethanol wash and elution into TE (10 mM Tris-HCl, 1 mM EDTA, pH 8.0). Approximately 300 ng of eluted DNA served as the template in separate gene- and pseudogene-specific LR-PCR reactions with the following final concentrations: 1×LongAmp Taq Reaction Buffer (New England Biolabs, NEB), 0.3 mM dNTPs, 1 μM of a gene- or pseudogene-specific forward primer, 1 μM of common reverse primer LRPCR_Unv_R (all primer sequences in Table S3 of Appendix), 0.25% Formamide, and 5 units LongAmp Hot Start Taq DNA Polymerase (NEB). Reactions including the gene-specific forward primer PMS2_LRPCR_F yielded a ˜17 kb amplicon spanning PMS2 exons 11-15 (the forward primer targets exon 10), whereas use of the pseudogene-specific forward primer PMS2CL_F amplified ˜18 kb from PMS2CL (spans region upstream of PMS2CL through exon 6). Thermal-cycling involved initial denaturation at 94° C. for 5 min followed by 30 cycles of 94° C. for 30 s and 65° C. for 18.5 min. Final elongation was 18.5 min at 65° C., followed by a 4° C. hold. Quality of LR-PCR amplicons was assessed using 0.5% agarose gel electrophoresis and quantification with the broad range Qubit assay kit (Thermo Fisher).

Two different library-prep strategies were used to prepare LR-PCR amplicons for NGS. In the first, applied to patient samples, LR-PCR amplicons were fragmented by adding 2 μL NEBNext dsDNA Fragmentase and NEBNext dsDNA Fragmentase Reaction Buffer v2 (1× final, NEB) to the remaining LR-PCR reaction volume, and then incubated at 37° C. for 25 min. Addition of 100 mM EDTA stopped the reaction, which underwent cleanup with 1.5×SPRI beads, followed by 80% ethanol wash and elution in TE. Fragmentation quality was assessed via Bioanalyzer (Agilent) with the High Sensitivity DNA kit. NGS library prep included end repair, A-tailing, and adapter ligation. Samples were PCR amplified with KAPA HiFi HotStart PCR Kit (Kapa Biosystems) for 8-10 cycles with barcoded primers with the following thermal cycling: initial denaturation at 95° C. for 5 min followed by cycles of 98° C. for 20 s, 60° C. for 30 s, and 72° C. for 30 s. The last elongation was 5 min at 72° C., followed by 4° C. hold. Library quality was verified via Bioanalyzer with a High Sensitivity DNA kit and the concentration was measured with absorbance via a microplate reader (Tecan Infinite M200 PRO).

The second approach to prepare LR-PCR amplicons for NGS—applied to the 155 cell-line samples—entailed fragmenting and inserting adapters into LR-PCR amplicons via tagmentation. Two duplex adapters were created by annealing single-stranded oligonucleotides: one duplex adapter had the Unv_Tn5_oligo (all primer sequences in Table S3) annealed to Oligo A; the other duplex adapter had the Unv_Tn5_oligo annealed to Oligo B. The two separate annealing mixes included 25 μM of each oligonucleotide in the duplex plus 1×annealing buffer (10 mM Tris-HCl, 50 mM NaCl, 1 mM EDTA, pH 8.0). The reaction was denatured at 95° C. for 2 min, incubated at 80° C. for 60 min, stepped down in temperature by 1° C. every minute until reaching 20° C., and then held at 4° C. Adapters were loaded into the Tn5 enzyme during a 30 min incubation at 37° C. with 0.15 units of Robust Tn5 Transposase (kit from Creative Biogene), 1.25 μM of each adapter, and 1×TPS buffer. LR-PCR amplicons were subjected to tagmentation with the Tn5-adapter construct. Tagmentation reactions occurred at 56° C. for 10 min in 1×LM Buffer, with 0.5 μL of loaded Tn5 and 1-2 ng of DNA from each LR-PCR reaction. After incubating, SDS (0.02% final) was added to each reaction and incubated for 5 min to dissociate Tn5 from the DNA. Tagmention cleanup with 1×SPRI beads preceded molecular barcoding and amplification via PCR to generate NGS libraries. The PCR reaction included 1 unit Kapa HiFi Polymerase (Kapa Biosystems), 1×HiFi Buffer, 375 μM dNTPs, 0.5 μM of each primer, and the cleaned-up tagmented sample. Cycling started with gap-filling at 72° C. for 3 min and followed with 10 cycles of denaturation at 98° C. for 30 s, annealing at 63° C. for 30 s, and extension at 72° C. for 3 min. Cleanup of NGS libraries was performed with 1×SPRI beads.

For patient samples, LR-PCR libraries were sequenced on a HiSeq 2500 (Illumina) in rapid run mode (paired reads, 150 cycles each). For cell line samples, LR-PCR libraries were sequenced on a NextSeq 550 (Illumina) to a minimum depth of 500 reads (single read, 150 cycles).

### Hybrid Capture and Sequencing

Targeted NGS was performed as described previously [7,8]. Briefly, DNA from a patient's blood or saliva sample was isolated, quantified by a dye-based fluorescence assay, and then fragmented to 200-1000 bp by sonication. Fragmented DNA was converted to an NGS library by end repair, A-tailing, and adapter ligation. Samples were then amplified by PCR with barcoded primers, multiplexed, and subjected to hybrid capture-based enrichment with 40-mer oligonucleotides (Integrated DNA Technologies) complementary to regions common between PMS2 and PMS2CL. NGS was performed on a HiSeq 2500 with mean sequencing depth of ˜500× for the whole panel (coverage in PMS2 is ˜1000×). All target nucleotides are required to be covered with a minimum depth of 20 reads.

### Read Alignment

For hybrid-capture data, in order to aggregate PMS2- and PMS2CL-originating reads at the PMS2 locus in the reference genome, paired-end NGS reads were first aligned to the hg19 human reference genome using BWA-MEM [27]. The alignment at PMS2 exon 11 was filtered to only include reads that overlapped with a site of known difference between gene and pseudogene. Reads that aligned to PMS2 exons 12-15 and reads that aligned to PMS2CL exons 3-6 were partitioned into a BAM file using samtools [28]. The BAM file was converted into two unaligned FASTQ files (each member of the read pair parsed to one of the two files) using Picard (Broad Institute). Each single-end FASTQ file was separately realigned to the hg19 genome allowing for ambiguous alignments and reporting of the top several alignments for each read. The resulting single-end alignments were used to generate a paired-end alignment in the following manner: 1) both single-end reads had the same read name; 2) both single-end reads mapped to the region spanning PMS2 exons 12-15; 3) both single-end reads aligned within 1000 bp of each other, and 4) when multiple putative pairs met the above conditions for a given read name, the pair with the highest alignment score was chosen. Reads that could not form proper pairs as described above were discarded. The resulting paired-end BAM file contained reads originating from both PMS2 and PMS2CL mapped to the PMS2 sequence.

For RT-PCR data (described below) and LR-PCR data, NGS reads were aligned to the hg19 genome sequence in which the PMS2CL sequence was removed, thereby aggregating genic and pseudogenic reads in PMS2.

### SNV and Indel Calling

For the PMS2 region into which reads from PMS2 and PMS2CL were mapped (see above), SNVs and short indels were identified using GATK 4.0 HaplotypeCaller [29] with the sample-ploidy option set to four, the max-reads-per-alignment-start option off, and the min-pruning option set to one. For the diploid PMS2 exon 11 region, SNVs and short indels were identified using GATK 1.6 [30] and FreeBayes [31]. For diploid SNV calling in the LR-PCR data, GATK 1.6 was similarly used. For the LR-PCR sample in which we suspected allelic dropout (see Discussion), AB was determined by visual inspection of the NGS data in the Integrative Genomics Viewer [32].

### CNV Calling

For short-read NGS data of hybrid-captured fragments, CNVs in PMS2 exon 11 were determined by measuring the relative NGS read depth at target positions using the algorithm described previously [7]. To call CNVs in PMS2 exons 12-15 from BAM files in which PMS2- and PMS2CL-originating reads were positioned in the PMS2 sequence (see “Read Alignment” above), two modifications to the CNV calling algorithm were made: 1) the expected wild type copy number was changed from two to four copies, and 2) p_{CNV}, the parameter determining how likely the HMM is to transition from a wild-type to a CNV state, was set to 0.01 to obtain high CNV sensitivity and specificity from empirical data.

For CNV calling from LR-PCR data, read depth was counted in equal-sized bins (50 bp) that tile the amplicon. Bin counts for each sample were normalized by the median bin depth of the sample; next, each bin's values were normalized by the median of the bin. The same bins were used for corresponding regions of PMS2 and PMS2CL. The resulting binned and normalized data were searched for CNVs using the algorithm described previously [7]. CNV no-calls were manually reviewed to resolve status as positive or negative.

### CNV Simulations

Single-copy duplications and deletions were introduced by modifying the number of observed reads in one of the CNV-negative samples in a given batch of samples, as described previously [33]. For PMS2 exons 12-15, where baseline copy-number was four, single-copy deletions and duplications were introduced by subsampling reads to 75% or scaling read number by 125%, respectively. Simulated CNVs were created for every possible contiguous combination of exons in the last 4 exons in PMS2. For each CNV size and position, 2186 samples were simulated and tested via the CNV calling algorithm, and sensitivity was calculated as the percentage of the synthetic CNVs that were correctly detected. CNVs were simulated separately in PMS2 exon 11, which had a baseline copy number of two, because pseudogenic reads were filtered from the genic sequence.

### Tetraploid Indel Simulations

Indels in a tetraploid background (relevant for exons 12-15 of PMS2, where gene- and pseudogene-originating reads were remapped) were simulated to better test indel-calling sensitivity using GATK4. Two diploid alignments, at least one of which was previously determined via the Counsyl Reliant HCS panel to contain an indel, were merged to create a tetraploid alignment. If one of the samples had more reads than the other in the 100 bp region centered on the indel, reads were binomially downsampled such that each merged diploid sample had approximately the same number of aligned reads. Indels were then called from these synthetic tetraploid alignments using GATK4 as described in section SNV and Indel Calling above.

### Variant Curation

For all variants in the last five exons of PMS2, variant interpretation was performed in accordance with American College of Medical Genetics and Genomics (ACMG) criteria using a 5 tier classification category system (Benign, Likely Benign, Variant of Uncertain Significance, Likely Pathogenic, Pathogenic) [34]. Classifications were made using evidence available in the published literature and publicly available databases. Allele-frequency based rules were not used because of potentially inaccurate PMS2 variant identification in population databases. Variant classifications were reviewed and approved by board-certified laboratory directors.

### MLPA

MLPA was performed according to manufacturer's protocol (MRC Holland, probemix P008-C1 PMS2 protocol issued Dec. 11, 2017 and MLPA General Protocol issued on Mar. 23, 2018). Generally, genomic DNA was covered with mineral oil to reduce evaporation during hybridization and ligation; next, DNA was denatured for 5 min at 98° C. and then held at 25° C. Hybridization reagents and probemix were added to the samples and incubated at 95° C. for 1 min followed by 16-20 h at 60° C. Probe pairs that bind target DNA at adjacent positions were ligated for 15 min at 54° C. and then amplified via PCR for 35 cycles. Amplified probes were mixed with ROX ladder and formamide and then separated on a capillary electrophoresis instrument. Coffalyser software (MRC Holland) normalized PMS2 probe intensities to those of the reference probes first within each sample and then among samples. Normalized probe intensities of each sample were compared to the average intensities of the reference samples; Coffalyser emitted CNV calls in the region.

### Reflex Rate Estimate

The reflex rate was estimated using SNV-, indel-, and CNV-specific reflex rates from the LR-PCR and hybrid-capture data and subsequently extrapolating to a large cohort size using Markov Chain Monte Carlo simulations with pymc [35].

### Distinguishing Base Analysis

NGS reads from LR-PCR amplicons from PMS2 and PMS2CL were aligned to PMS2, and variants were called with GATK UniversalGenotyper. Sites were considered reliable if variants were homozygous for the reference allele in the PMS2-specific amplicon and homozygous for an alternate allele in the PMS2CL-specific amplicon (as aligned to PMS2) in 100% of samples.

### RNA Testing

### RNA Extraction and Reverse Transcription:

RNA was extracted from 33 samples with the Agencourt RNAdvance Blood kit (Beckman Coulter) from 400 μL of whole blood following the manufacturer's instructions. RNA was extracted from blood tubes no more than seven days after blood draw was performed. Extraction quality was assessed with the RNA 6000 Nano kit (Agilent). RNA was quantified with Qubit HS RNA Assay kit (Thermo Fisher).

RNA was reverse transcribed using Superscript II Reverse Transcriptase with oligo-dT and random hexamers as primers (kit from Thermo Fisher). Reactions were performed as follows: 0.1-1.0 μg total RNA, 1.25 μM of both random hexamers and oligo-dT primer, 0.8 mM dNTPs, and water up to a final volume of 12 μL. Reactions were heated at 65° C. for 5 min and then chilled on ice for 5 min. 1×first-strand buffer and 0.01 M DTT were added to each reaction and incubated at 42° C. for 2 min. 10 U/μL Superscript II Reverse Transcriptase was added to each reaction and incubated at 42° C. for 50 min, then heat inactivated at 72° C. for 15 min. A positive control of pooled mRNA (Stratagene, Catalog #750500-41) was used with each reverse transcription reaction.

Following reverse transcription, RNA was hydrolyzed with 2 μL 1N NaOH and heated at 95° C. for 5 min. 4 μL of 1 M Tris-HCL pH 7.5 was used to neutralize the reaction for downstream processing. Qubit ssDNA Assay kit (Thermo Fisher) was used to quantify cDNA.

### PCR:

For each sample, two reactions were set up: 1) forward primer PMS2_RNA_F and reverse primer RNA_Unv_R amplified 1.5 kb of PMS2 from cDNA and 2) forward primer PMS2CL_F and reverse primer RNA_Unv_R amplified 1.5 kb of PMS2CL from cDNA (primer sequences in Table S3 of Appendix). PCR reactions contained 1×LongAmp Taq Reaction Buffer (NEB), 0.3 mM dNTPs, 1 μM of each forward and reverse primer, 20-70 ng cDNA, 0.1 U/μL LongAmp Taq DNA polymerase (NEB), and water up to 25 μL. Thermocycling was as follows: 94° C. for 5 min, 30 cycles of 94° C. for 30 s, annealing at 52° C. for PMS2 and 55° C. for PMS2CL, 65° C. for 2 min, followed by a final extension at 65° C. for 10 min and then a 4° C. hold. PCR products were cleaned with 1.2×SPRI beads. Amplicons were visualized with a 2% agarose gel or with the DNA 7500 kit (Agilent).

### Sequencing:

50-100 ng of each amplicon were fragmented in 50 μL volumes with a Bioruptor (Diagenode) for 12 cycles, 30 s on and 90 s off. Fragmentation was visualized with High Sensitivity DNA kit (Agilent). All fragmented material was used as input for library preparation. KAPA Hyper Prep kit (Kapa Biosystems) was used for library preparation, and manufacturer instructions were followed. Adapters were diluted to 15 μM for PMS2 and 3 μM for PMS2CL. Nine cycles of enrichment PCR were performed. Samples were quantified using absorbance measurements (Tecan M200), normalized to 10 nM, and consolidated into one reaction. The final library was quantified with qPCR using KAPA Library Quantification Kit (Kapa Biosystems) and sequenced on the NextSeq 550 System (Illumina) for 75 cycles single read with dual indexing.

### Alignment:

Basecall files were converted to FASTQ files using bcl2fastq (Illumina). FASTQ files were aligned using STAR [36].

### Analytical Metrics

Metrics were defined as follows: Sensitivity=TP/(TP+FN); Specificity=TN/(TN+FP). The CIs were calculated by the method of Clopper and Pearson [37]. For SNVs and indels, true negatives were defined as concordant negative results observed at sites found to be polymorphic in the cohort used (positions at which we observed non-reference bases in at least one sample).

### Results

Zero Nucleotides can Reliably Distinguish Exons 12-15 of PMS2 from PMS2CL:

NGS of short DNA fragments would only be able to identify PMS2-specific variants in the last five exons if the fragments themselves could be unambiguously aligned to the gene or pseudogene. To overcome pseudogene interference, unique mapping would rely on the bases that differ between PMS2 and PMS2CL. In the hg19 reference genome, these distinguishing bases are scarce (FIG. 1D, light bars): sequence identity in each of the last five exons of PMS2 (padded with 20 nt of intronic sequence) exceeds 97%, and the differences comprise only 26, 0, 1, 1, and 0 bases in exons 11 through 15, respectively. Further, previous reports noted that natural variation may suppress the reliability of these distinguishing bases represented in the reference genome [17,18].

To test the reliability of the reference genome, a catalog of natural variation in PMS2 exons 11-15 and the corresponding regions in PMS2CL was assembled. NGS was performed on gene- and pseudogene-specific LR-PCR amplicons on 707 of the patient samples in the cohort used (Table 1) with diverse self-reported ethnicities (Table S4 of Appendix). It was found that 7 of the 26 expected positions in PMS2 exon 11 had distinct alleles in the gene and pseudogene, making them reliable distinguishing bases. In contrast, for 19 positions in exon 11 and two positions in exons 12-15, the ostensibly PMS2-specific alleles from hg19 were observed at least once in the PMS2CL LR-PCR data, and vice versa (see Table S4 of Appendix for allele frequencies). Therefore, after accounting for the natural variation in gene and pseudogene, there are zero reliable distinguishing bases (i.e., 100% sequence identity) in PMS2 exons 12-15, and seven distinguishing bases in exon 11 (FIG. 1D, dark bars). Together, these data suggest that variant identification via short-read NGS alone could be sufficient for exon 11, but a different approach is required for exons 12-15.

TABLE 1
Summary of Samples

| Assay | Samples |
|---|---|
| LR-PCR + NGS | 718 patient samples |
| | 155 cell-line samples |
| Hybrid capture + NGS | 144 patient samples |
| | 155 cell-line samples |
| | 3 known positives |
| MLPA | 4 patient samples |
| | 4 cell-line samples |
| RT-PCR + NGS | 33 samples |

Reflex Workflow to Disambiguate Variants Discovered with Short-Read NGS:

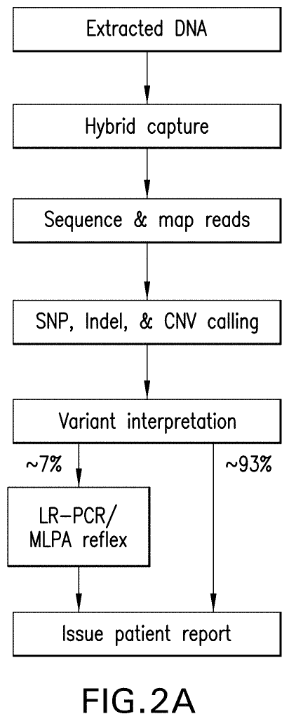

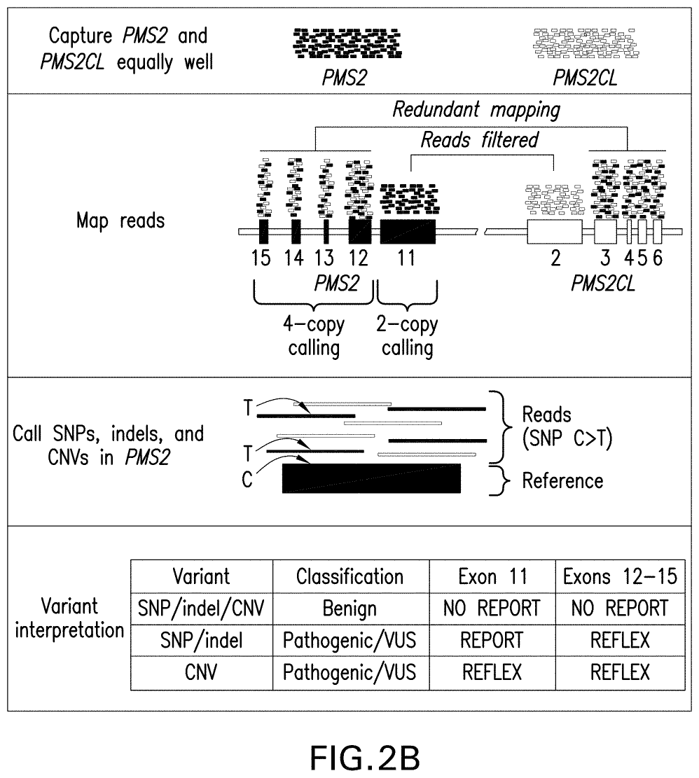

The plausibility of a workflow for the 3′ exons of PMS2 was evaluated, that uses short-read NGS as its foundation and performs reflex testing with orthogonal assays to disambiguate the genic or pseudogenic origin of variants only when clinically needed (FIG. 2A). In the short-read NGS stage of testing, the molecular approach is consistent across the last five exons of PMS2: DNA fragments are captured in a manner that is agnostic to their genic or pseudogenic origin by designing capture probes that specifically avoid positions shown to vary between PMS2 and PMS2CL in the LR-PCR data from patient samples (FIG. 2B, purple box).

The workflow employs different bioinformatics strategies for PMS2 exon 11 and for the group of exons 12-15 (FIG. 2B, blue box). For exon 11, PMS2-specific variants are identified by tailoring the read-alignment software to partition reads to PMS2 or PMS2CL based on the gene- and pseudogene-distinguishing bases. By contrast, for PMS2 exons 12-15, reads are aligned with permissive settings such that each read will align to both its best genic location and its best pseudogenic location (see Methods). For the typical sample with two copies each of PMS2 and PMS2CL, this approach effectively provides read depth in each location corresponding to four copies. To identify SNVs, indels, and CNVs, the variant calling software is adjusted such that it anticipates a baseline ploidy of two in exon 11 and four in exons 12-15 (FIG. 2B, blue and green boxes).

Disambiguation via reflex testing is only required for a subset of variants based on their type and clinical interpretation (FIG. 2B, orange box). As such, variant interpretation is performed prior to reflex testing. Benign variants are not reflex tested or reported to patients. Samples with CNVs in any of the last five exons of PMS2 that are classified as pathogenic, likely pathogenic, or variants of uncertain significance (VUS) undergo reflex testing for disambiguation. Samples with non-benign SNVs or indels in exons 12-15 are reflex tested for disambiguation, but samples with such variants in exon 11 are simply reported without reflex due to unique read mapping in that exon. Disambiguation testing for SNVs, indels, and CNVs can be performed via LR-PCR followed by sequencing to determine if the variant came from PMS2 or PMS2CL; MLPA can assist resolution of CNVs [20].

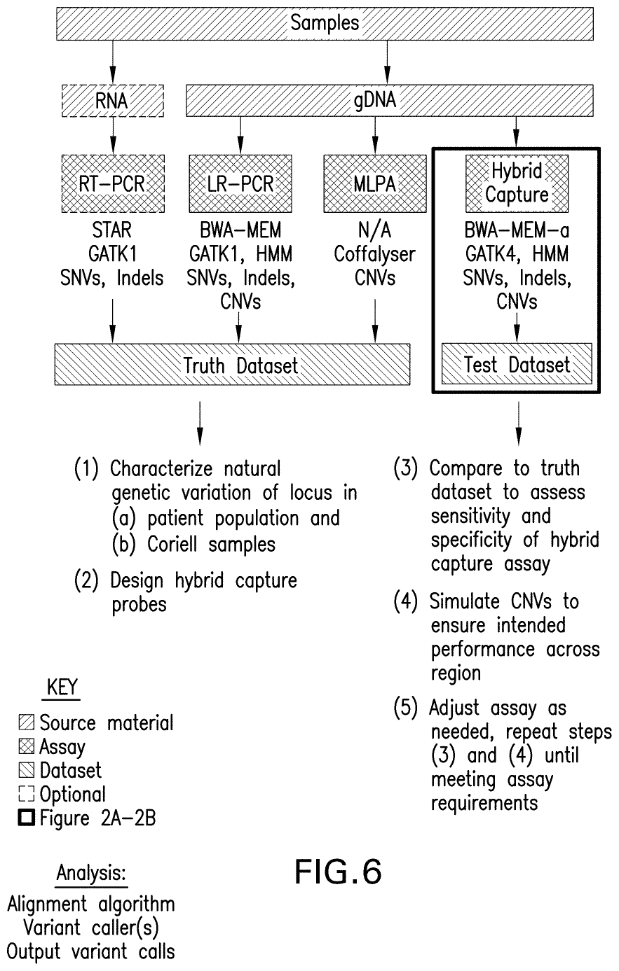

Executing the proposed workflow resolves cancer risk associated with the last five exons of PMS2 for the majority of samples with short-read NGS alone. For each of the 707 patient samples that underwent LR-PCR (Table 1), variant classification was performed on the results and found that nearly 93% could forgo reflex testing. The remaining ˜7% would have required subsequent testing to yield confident PMS2 screening results (FIG. 2A). The SNV- and indel-specific component of this reflex rate was 41/707 (5.8%), and the reflex rates due to CNV calls and no-calls were 2/707 (0.3%) and 1/144 (0.7%), respectively. Using simulations (see Methods), the reflex rate on a larger cohort of 13,000 patients was estimated to be 7.7% (95% CI: 5.4-10.7%). The 0.7% contribution to the reflex rate from samples with CNV no-calls is expected to be an upper-bound estimate because a standard practice of retesting such samples at least once on short-read NGS typically yields a confident negative call (data not shown), thereby avoiding reflex testing. Therefore, the overall reflex rate of the proposed workflow (see FIG. 6) is anticipated to be less than 8%.

### Short-Read NGS Accurately Identified Samples Needing Reflex Testing for SNVs and Indels:

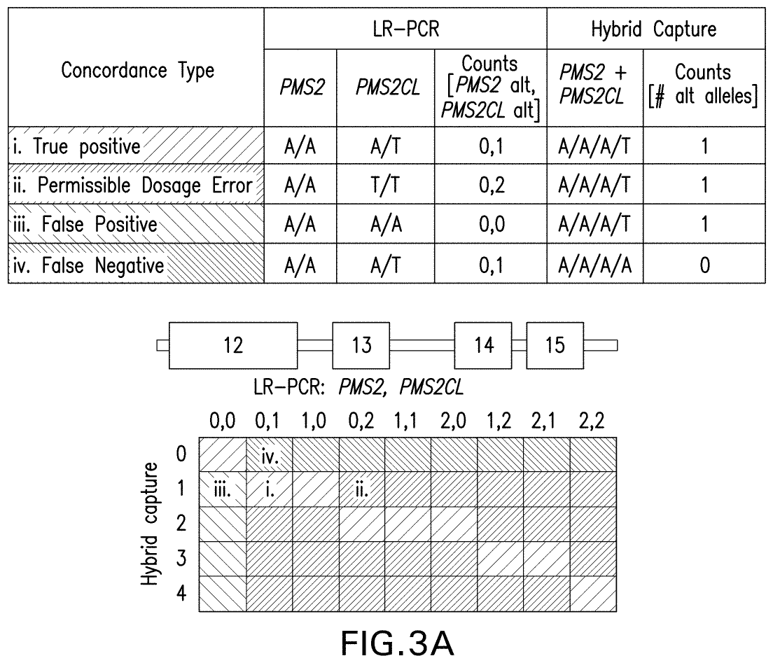

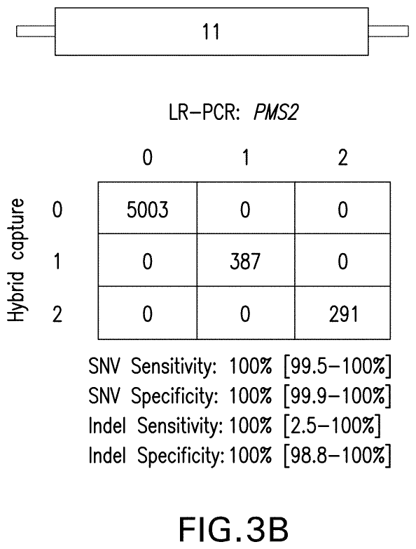

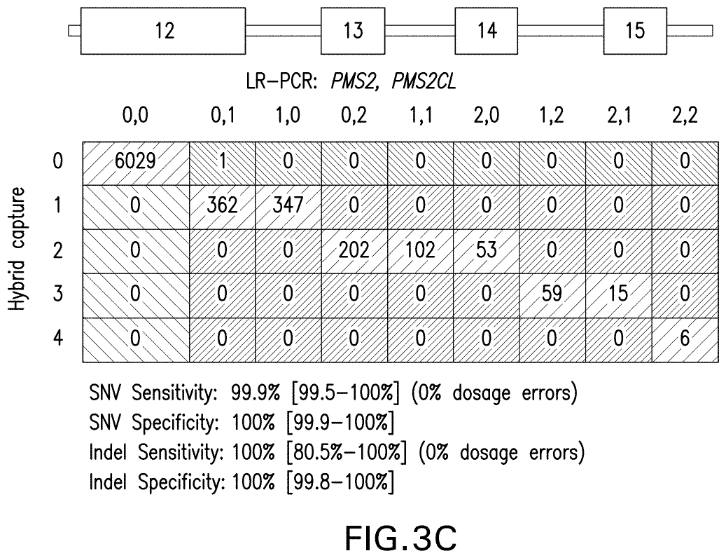

The reflex workflow described herein is only clinically viable if the short-read NGS test (FIG. 2B) has high analytical sensitivity and specificity for (1) identifying variants in PMS2 exon 11 and (2) flagging samples that need reflex testing for variants in exons 12-15 with ambiguous PMS2/PMS2CL origin. To evaluate accuracy of the short-read NGS testing for SNVs and indels, its results were compared to those observed with LR-PCR for 144 patient samples and 155 cell lines (FIG. 3). Measuring genotype concordance in exons 12-15 required an atypical confusion matrix because short-read NGS genotypes were reported as tetraploid (see Methods), whereas the LR-PCR returned diploid genotype calls for both the gene and pseudogene (FIG. 3A highlights several examples). The matrix includes “Permissible Dosage Errors,” where the presence of alternate alleles is properly detected but the number of alternate alleles is discordant; such errors are deemed permissible because the presence of alternate alleles in short-read NGS would suffice to trigger reflex testing and be corrected. When compared at 1,678 sites with LR-PCR as a truth set, short-read NGS testing had 100% analytical sensitivity and 100% analytical specificity in exon 11 (FIG. 3B), and 99.9% analytical sensitivity and 100% analytical specificity in exons 12-15 (FIG. 3C).

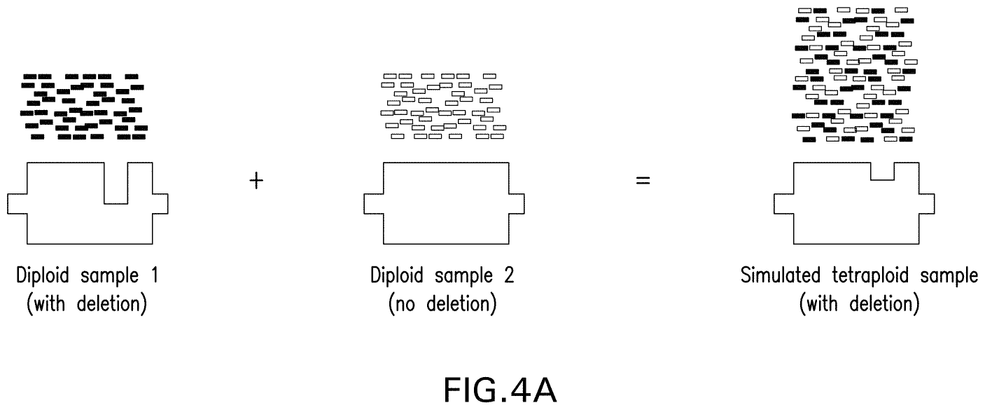

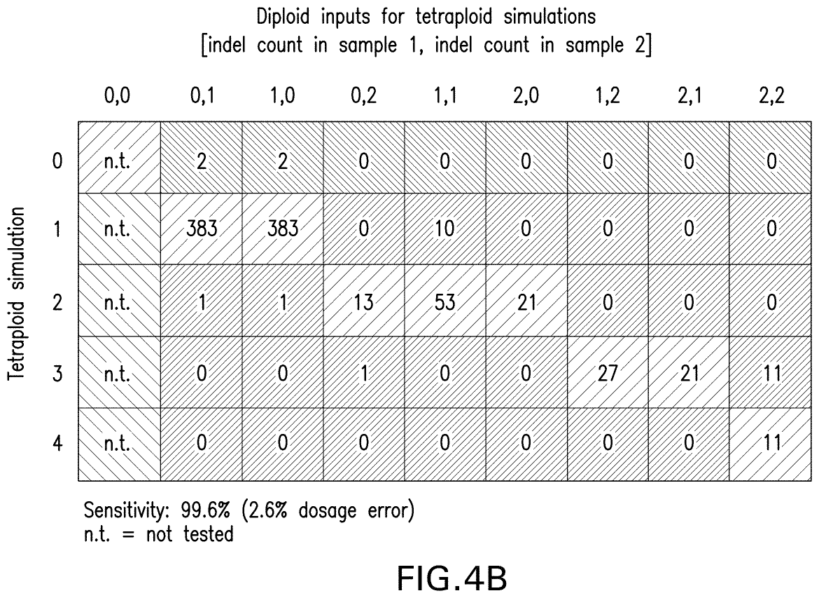

The scarcity of indel calls in the patient cohort used and cell lines (17 overall)—coupled with the uncommon usage of variant-calling software in a tetraploid-background mode for a clinical genomics application—motivated a deeper examination of indel-calling efficacy in PMS2 exons 12-15. The expected NGS data was simulated for samples with a tetraploid genome background populated with indels of different allele dosages (1, 2, 3, or 4 copies). To construct such samples, the diploid NGS data were merged from two samples (at least one containing an indel) in a region of the HCS test used other than PMS2 (FIG. 4A, see Methods). The respective genotypes of the two samples provided an expected genotype of the merged sample: for instance, combining a heterozygous sample (one indel allele) with a homozygous-alternate sample (two indel alleles) would give an expected indel dosage of three. FIG. 4B illustrates 99.6% sensitivity for indels in the simulated tetraploid background, suggesting that sensitivity is comparably high in exons 12-15 in PMS2 where the read-alignment and variant-calling strategy used yields a tetraploid background. Because the empirical data in FIG. 3C demonstrate 100% specificity for indels in exons 12-15, specificity was not further evaluated with simulations.

In sum, a comparison of SNV and indel calls between LR-PCR and short-read NGS suggests the pre-reflex step of the proposed workflow described herein achieves sufficient analytical sensitivity and specificity to be considered for clinical use.

Accurate Detection with Short-Read NGS of Samples Needing CNV Reflex Testing:

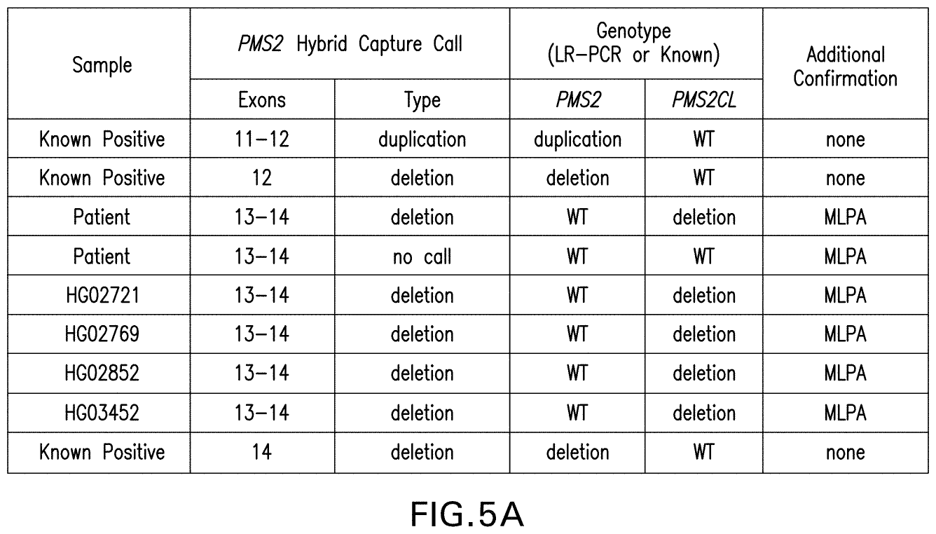

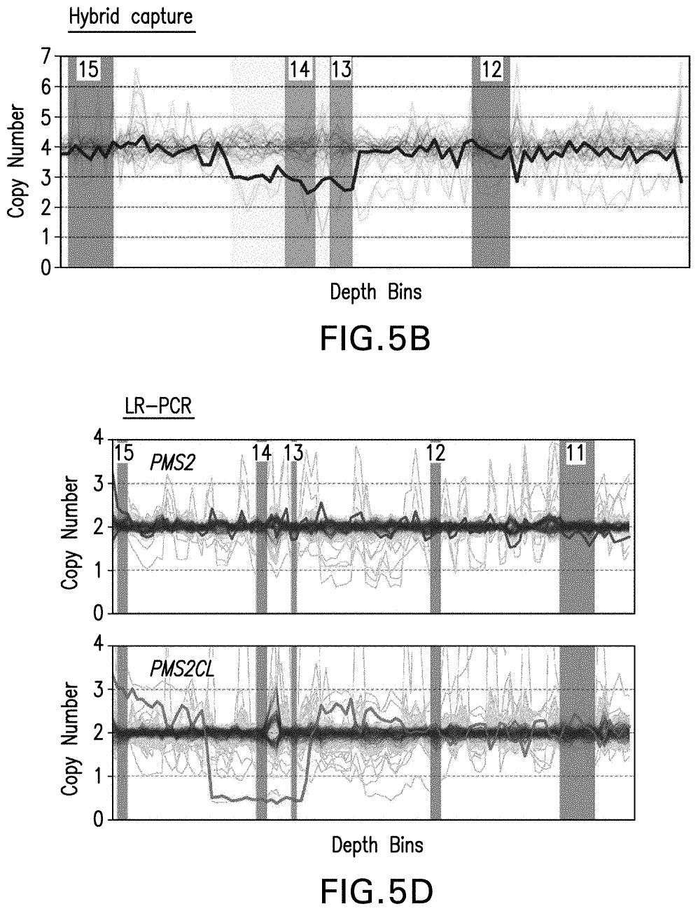

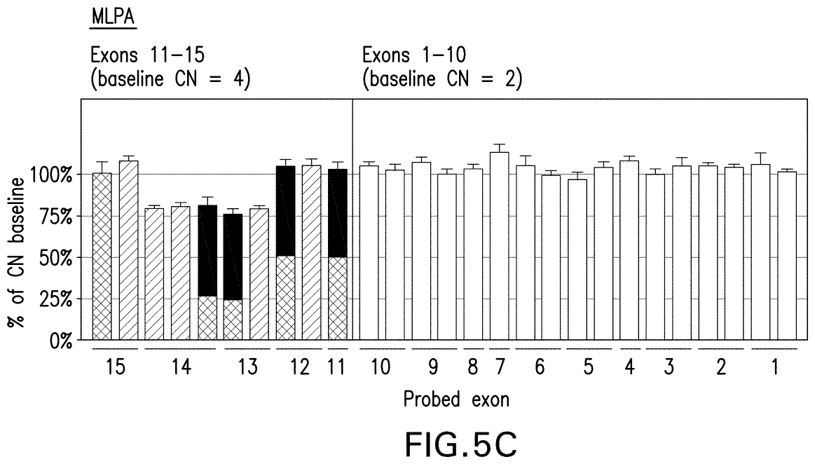

To evaluate the sensitivity and specificity of short-read NGS for CNVs in the last five exons of PMS2, patient samples, cell lines, known positives, and samples with simulated positives were tested. As with SNVs and indels, the CNV detection algorithm described above was adapted to use a copy-number baseline of two for PMS2 exon 11 and four for exons 12-15 (FIG. 2B, blue box; see Methods). The three known-positive samples with CNVs in the last five exons were correctly identified as harboring CNVs encompassing the expected exons (FIG. 5A). A deletion of exon 13-14 in four of the cell lines and one of the clinical samples was additionally observed; for the clinical sample, short-read NGS identified a drop in signal from the tetraploid background (FIG. 5B), MLPA confirmed the presence of a similar deletion (FIG. 5C), and NGS on the LR-PCR amplicons revealed that the deletion was in PMS2CL rather than PMS2 (FIG. 5D). Interestingly, though only one of two copies of this region is deleted in PMS2CL, the LR-PCR profile shows a 75% signal drop in the deleted region. It is speculated that this arises from preferential amplification of the shorter deletion-harboring allele during LR-PCR. Therefore, although the LR-PCR data were unique in providing disambiguation, the short-read NGS and MLPA data had more readily interpretable copy-number values.

Due to the absence of a large catalog of CNV-positive samples, thorough and direct characterization of PMS2 CNV calling sensitivity with short-read NGS would require blind testing of thousands of samples. Instead, sequencing data from the abundance of CNV-negative patients was used as substrate in simulations that introduce CNVs of given length and location (see Methods). By running the CNV detection algorithm described above on the 2186 simulated samples, the analytical sensitivity for CNVs ranging from one to five exons in length was measured (Table 2; simulation data on cell-line samples in Table S6 of Appendix).

Sensitivity for multi-exon deletions generally exceeded 99.2% and for single-exon deletions was ˜89%. Weighing the simulated sensitivities by the observed frequency distribution of CNV length in the last five exons of PMS2 [21,23,24], it is estimated that aggregate CNV sensitivity in this complicated genomic region is 96.7%.

TABLE 2
CNV simulations demonstrate high analytical sensitivity

| | Deletions | | | | | Duplications | | | | | | Overall |
|---|---|---|---|---|---|---|---|---|---|---|---|---|
| | Size (exons) | | | | | | | | | | | Sensitivity |
| | 1 | 2 | 3 | 4 | 5 | 1 | 2 | 3 | 4 | 5 | Exon 11-12 | (weighted) |
| Sensitivity | 88.9% | 99.2% | 100% | 100% | 100% | 70.0% | 93.8% | 99.3% | 100% | 100% | 100% | 96.7% |
| Weights | 26% | 21% | 8% | 15% | 26% | 0% | 4% | 0% | 0% | 0% | n/a | |

High sensitivity for CNVs must not come at the expense of low specificity, prompting measurement of the CNV false-positive rate in the large cohort used. In the 302 hybrid capture cohort of 302 samples, there was one no-call, which is treated as a false positive. Therefore, sample-level specificity is 99.7% (95% CI: 98.2-100%).

Based on these analyses, it is concluded that short-read NGS—as optimized in the described workflow—can achieve >96% sensitivity and >99% specificity for detecting samples with CNVs in the terminal five exons of PMS2.

### Gene- and Pseudogene-Specific Variant Information for Common Cell Lines:

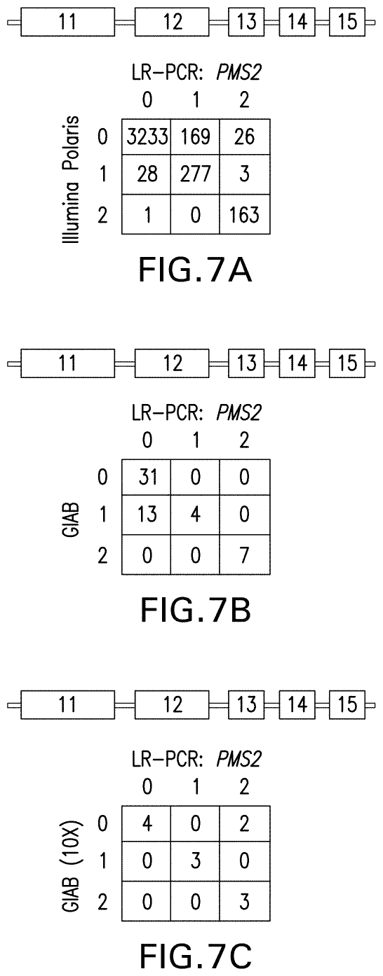

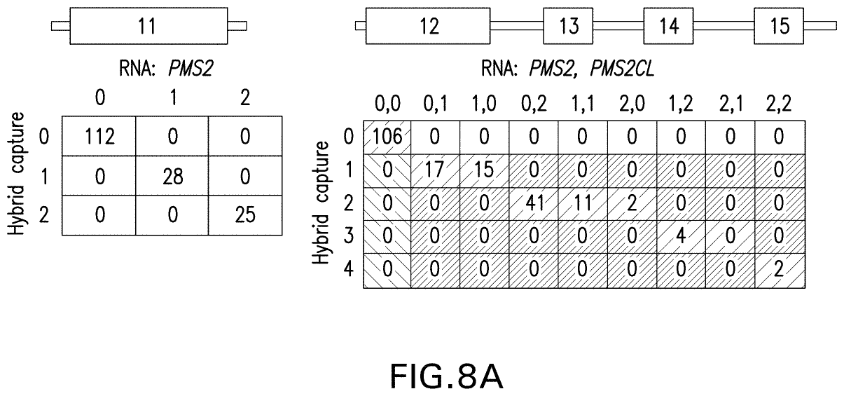

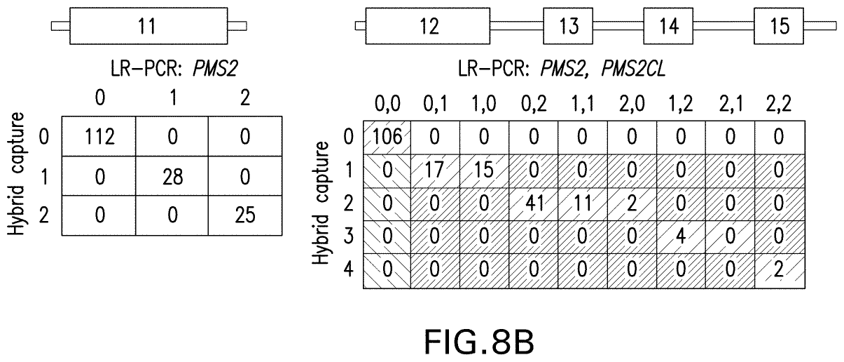

Reference cell lines with known genotypes facilitate development and validation of novel molecular diagnostic methods, yet samples with high-quality genotypes in the PMS2 region are generally unavailable due to the region's complicated nature. In the course of developing and testing the workflow characterized above, NGS was performed of both hybrid-capture fragments and LR-PCR amplicons on cell lines where high-quality genome sequences were assembled from whole-genome sequencing with ˜30× depth (Illumina Polaris 1 Diversity Panel) or from the Genome in a Bottle (GIAB) Consortium [38,39]. Importantly, FIG. 7 shows that the gene-specific genotypes observed differed from the Polaris and GIAB data (including phased data on GIAB samples; FIG. 7C). In principle, such differences could arise due in part to errors in either dataset, for example through biological contamination, non-specific amplification, non-specific sequence alignment, or technical processing errors by the chosen genotyping software. The concordance between orthogonal hybrid-capture and LR-PCR assays suggests that the genotypes reported here are correct, but as a third orthogonal method, PMS2 and PMS2CL were also genotyped from RNA extracted from 33 of the LR-PCR samples (see Methods). The RNA-derived genotypes were concordant with the LR-PCR data (FIG. 8), strongly suggesting that we elucidated correct gene- and pseudogene-specific genotypes. To aid scientific research and clinical development of PMS2 and its role in Lynch syndrome, the gene- and pseudogene-specific variant information are shared. For patient samples, to share valuable data while being mindful of patient consent and PHI compliance, variant frequencies are provided (Table S4 of Appendix). For cell lines, variant frequencies are shared, as well as BAM and VCF files for the LR-PCR amplicons spanning the last five exons of PMS2 and PMS2CL (Table S5 of Appendix and in ENA accession #PRJEB27948).

### EXEMPLARY EMBODIMENTS

The following embodiments are exemplary and are not intended to limit the present invention.

Embodiment 1. A method for detecting genetic variation in a genome of a subject, the genome comprising highly homologous first and second regions of interest, the method comprising:

(a) obtaining sequence reads by paired-end sequencing from multiple sites of interest in the first and second regions of interest, wherein the sequence reads comprise a first read and a second read obtained at each site of interest;

(b) aligning sequence reads to a reference genome, wherein first reads and second reads are aligned to the reference genome separately and the aligner emits multiple possible alignments for each of the first and second reads;

(c) identifying first reads and second reads that align to the first region of interest;

(d) pairing a first read and a second read from the reads identified in step (c), thereby generating a top paired alignment; and

(e) detecting the genetic variation in the top paired alignment generated in step (d).

Embodiment 2. The method of embodiment 1, comprising, before step (b), aligning first reads and second reads to a reference genome, wherein the aligner emits the best possible paired-end alignment to the first or second region of interest for each pair of first and second reads, and wherein only paired-end reads associated with a top alignment score to the first or second regions of interest are aligned separately in step (b).

Embodiment 3. The method of embodiment 1, wherein the sequence reads are obtained by direct targeted sequencing (DTS) of the multiple sites of interest, and wherein the first read comprises a genomic sequence read and the second read comprises a probe sequence read associated with a site of interest.

Embodiment 4. The method of embodiment 1, wherein in step (b) the sequence reads are aligned using the Burrows-Wheeler Aligner (BWA) algorithm.

Embodiment 5. The method of embodiment 1, wherein in step (b) the aligner only emits alignments that meet a minimum alignment score for the first and second regions of interest.

Embodiment 6. The method of embodiment 1, wherein a first read and a second read are paired in step (d) only if the alignments of the first read and the second read to the first region of interest are within a certain number of bases of each other.

Embodiment 7. The method of embodiment 1, wherein a first read and a second read are paired in step (d) only if the alignments of the first read and the second read to the first region of interest are within about 100 bp, about 200 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp, about 1500 bp, or more than 1500 bp.

Embodiment 8. The method of embodiment 1, comprising generating multiple paired alignments in step (d), calculating an alignment score for each of the multiple paired alignments, and identifying the top paired alignment as having the highest alignment score.

Embodiment 9. The method of embodiment 1, wherein the top paired alignment in step (d) is selected as having the smallest template length.

Embodiment 10. The method of embodiment 1, wherein the genetic variation comprises SNPs, indels, inversions, and/or CNVs.

Embodiment 11. The method of embodiment 1, wherein the detecting in step (e) comprises calling SNPs, indels, inversions, and/or CNVs.

Embodiment 12. The method of embodiment 1, wherein the detecting in step (e) comprises using a hidden Markov model (HMM) caller to determine a copy number.

Embodiment 13. The method of embodiment 1, wherein the detecting in step (e) is based on an expected ploidy of 2.

Embodiment 14. The method of embodiment 1, wherein the detecting in step (e) is based on an expected ploidy of 4.

Embodiment 15. The method of embodiment 1, wherein if a genetic variation is detected in step (e), a portion of the subject's genome is amplified by long-range PCR and assayed by multiplex ligation-dependent probe amplification (MLPA).

Embodiment 16. The method of embodiment 1, wherein if a genetic variation is detected in step (e), a portion of the first region of interest is amplified by long-range PCR and the product or a portion thereof is sequenced by Sanger sequencing or NGS.

Embodiment 17. The method of embodiment 1, wherein if a genetic variation is detected in step (e), the subject's genomic DNA is assayed by multiplex ligation-dependent probe amplification (MLPA).

Embodiment 18. The method of embodiment 1, wherein the sequence reads are 30-50 bp or 100-200 bp in length.

Embodiment 19. The method of embodiment 1, wherein the highly homologous first and second regions of interest are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more than 99% identical.

Embodiment 20. The method of embodiment 1, wherein the sequence reads are obtained from one or more exons within the first and/or second region(s) of interest.

Embodiment 21. The method of embodiment 1, wherein the sequence reads are obtained from one or more introns within the first and/or second region(s) of interest.

Embodiment 22. The method of embodiment 1, wherein the sequence reads are obtained from one or more exons and introns within the first and/or second region(s) of interest.

Embodiment 23. The method of embodiment 1, wherein the sequence reads are obtained from one or more exons and introns within the first and/or second region(s) of interest, and wherein the introns are near the exons.

Embodiment 24. The method of embodiment 1, wherein sequence reads are obtained from one or more clinically actionable regions associated with the first and/or second region(s) of interest.

Embodiment 25. The method of embodiment 1, wherein the first region of interest comprises a gene and the second region of interest comprises a pseudogene.

Embodiment 26. The method of embodiment 1, wherein the first region of interest comprises a pseudogene and the second region of interest comprises a gene.

Embodiment 27. The method of embodiment 1, wherein the first region of interest comprises two alleles.

Embodiment 28. The method of embodiment 1, wherein the second region of interest comprises two alleles.

Embodiment 29. The method according to any one of embodiments 25-28, wherein the gene is PMS2.

Embodiment 30. The method according to any one of embodiments 25-28, wherein the pseudogene is PMS2CL.

Embodiment 31. The method of embodiment 1, wherein the multiple sites of interest are within an exon of PMS2 and an exon in another part of the subject's genome.

Embodiment 32. The method of embodiment 1, wherein the multiple sites of interest are within an exon of PMS2 and an exon of PMS2CL.

Embodiment 33. The method of embodiment 1, wherein the multiple sites of interest are within exons 11, 12, 13, 14, and/or 15 of PMS2 and exons 2, 3, 4, 5, and/or 6 of PMS2CL.

Embodiment 34. The method of embodiment 1, wherein the subject is a human and the sequence reads are aligned to a human reference genome.

Embodiment 35. The method of embodiment 1, wherein the method is computer-implemented.

Embodiment 36. The method of embodiment 1, wherein the reference genome does not comprise a masked or modified portion of a first or second homologous region of interest.

Embodiment 37. A non-transitory computer-readable storage medium comprising computer-executable instructions for carrying out embodiment 1.

Embodiment 38. A system comprising:

(a) one or more processors;

(b) memory; and

(c) one or more programs, wherein the one or more programs are stored in the memory and configured to be executed by the one or more processors, the one or more programs including instructions for carrying out embodiment 1.

### REFERENCES

1. Nagy R, Sweet K, Eng C. Highly penetrant hereditary cancer syndromes. Oncogene. 2004; 23: 6445-6470.
2. Lu K H, Wood M E, Daniels M, Burke C, Ford J, Kauff N D, et al. American Society of Clinical Oncology Expert Statement: collection and use of a cancer family history for oncology providers. J Clin Oncol. 2014; 32: 833-840.
3. Mucci L A, Hjelmborg J B, Harris J R, Czene K, Havelick D J, Scheike T, et al. Familial Risk and Heritability of Cancer Among Twins in Nordic Countries. JAMA. 2016; 315: 68-76.
4. Foulkes W D. Inherited Susceptibility to Common Cancers. N Engl J Med. 2008; 359: 2143-2153.
5. Garber J E, Offit K. Hereditary cancer predisposition syndromes. J Clin Oncol. 2005; 23: 276-292.
6. Vogelstein B, Papadopoulos N, Velculescu V E, Zhou S, Diaz L A, Kinzler K W. Cancer Genome Landscapes. Science. 2013; 339: 1546-1558.
7. Vysotskaia V S, Hogan G J, Gould G M, Wang X, Robertson A D, Haas K R, et al. Development and validation of a 36-gene sequencing assay for hereditary cancer risk assessment. PeerJ. 2017; 5: e3046.
8. Kang H P, Maguire J R, Chu C S, Haque I S, Lai H, Mar-Heyming R, et al. Design and validation of a next generation sequencing assay for hereditary BRCA1 and BRCA2 mutation testing. PeerJ. 2016; 4: e2162.
9. Bunnell A E, Garby C A, Pearson E J, Walker S A, Panos L E, Blum J L. The Clinical Utility of Next Generation Sequencing Results in a Community-Based Hereditary Cancer Risk Program. J Genet Couns. 2017; 26: 105-112.
10. Desmond A, Kurian A W, Gabree M, Mills M A, Anderson M J, Kobayashi Y, et al. Clinical Actionability of Multigene Panel Testing for Hereditary Breast and Ovarian Cancer Risk Assessment. JAMA Oncol. 2015; 1: 943-951.
11. Lynch H T, Smyrk T, Lynch J, Fitzgibbons R Jr, Lanspa S, McGinn T. Update on the differential diagnosis, surveillance and management of hereditary non-polyposis colorectal cancer. Eur J Cancer. 1995; 31A: 1039-1046.
12. Blount J, Prakash A. The changing landscape of Lynch syndrome due to PMS2 mutations. Clin Genet. 2018; 94: 61-69.
13. Sijmons R H, Hofstra R M W. Review: Clinical aspects of hereditary DNA Mismatch repair gene mutations. DNA Repair. 2016; 38: 155-162.
14. Tiwari A K, Roy H K, Lynch H T. Lynch syndrome in the 21st century: clinical perspectives. QJM. 2016; 109: 151-158.
15. Lynch H T, Fusaro R M, Lynch J F. Cancer Genetics in the New Era of Molecular Biology. Ann N Y Acad Sci. 1997; 833: 1-28.
16. De Vos M, Hayward B E, Picton S, Sheridan E, Bonthron D T. Novel PMS2 pseudogenes can conceal recessive mutations causing a distinctive childhood cancer syndrome. Am J Hum Genet. 2004; 74: 954-964.
17. Hayward B E, De Vos M, Valleley E M A, Charlton R S, Taylor G R, Sheridan E, et al. Extensive gene conversion at the PMS2 DNA mismatch repair locus. Hum Mutat. 2007; 28: 424-430.
18. van der Klift H M, Tops C M J, Bik E C, Boogaard M W, Borgstein A-M, Hansson K B M, et al. Quantification of sequence exchange events between PMS2 and PMS2CL provides a basis for improved mutation scanning of Lynch syndrome patients. Hum Mutat. 2010; 31: 578-587.
19. Vaughn C P, Robles J, Swensen J J, Miller C E, Lyon E, Mao R, et al. Clinical analysis of PMS2: mutation detection and avoidance of pseudogenes. Hum Mutat. 2010; 31: 588-593.
20. Vaughn C P, Hart K J, Samowitz W S, Swensen J J. Avoidance of pseudogene interference in the detection of 3′ deletions in PMS2. Hum Mutat. 2011; 32: 1063-1071.
21. van der Klift H M, Mensenkamp A R, Drost M, Bik E C, Vos Y J, Gille H J J P, et al. Comprehensive Mutation Analysis of PMS2 in a Large Cohort of Probands Suspected of Lynch Syndrome or Constitutional Mismatch Repair Deficiency Syndrome. Hum Mutat. 2016; 37: 1162-1179.
22. Li J, Dai H, Feng Y, Tang J, Chen S, Tian X, et al. A Comprehensive Strategy for Accurate Mutation Detection of the Highly Homologous PMS2. J Mol Diagn. 2015; 17: 545-553.
23. Vaughn C P, Baker C L, Samowitz W S, Swensen J J. The frequency of previously undetectable deletions involving 3′ Exons of the PMS2 gene. Genes Chromosomes Cancer. 2013; 52: 107-112.
24. Espenschied C R, LaDuca H, Li S, McFarland R, Gau C-L, Hampel H. Multigene Panel Testing Provides a New Perspective on Lynch Syndrome. J Clin Oncol. 2017; 35: 2568-2575.
25. Etzler J, Peyrl A, Zatkova A, Schildhaus H-U, Ficek A, Merkelbach-Bruse S, et al. RNA-based mutation analysis identifies an unusual MSH6 splicing defect and circumvents PMS2 pseudogene interference. Hum Mutat. 2008; 29: 299-305.
26. Herman D S, Smith C, Liu C, Vaughn C P, Palaniappan S, Pritchard C C, et al. Efficient Detection of Copy Number Mutations in PMS2 Exons with a Close Homolog. J Mol Diagn. 2018; 20: 512-521.
27. Li H. Aligning sequence reads, clone sequences and assembly contigs with BWA-MEM [Internet]. 2013. Available: arxiv.org/abs/1303.3997
28. Li H, Handsaker B, Wysoker A, Fennell T, Ruan J, Homer N, et al. The Sequence Alignment/Map format and SAMtools. Bioinformatics. 2009; 25: 2078-2079.
29. Poplin R, Ruano-Rubio V, DePristo M A, Fennell T J, Carneiro M O, Van der Auwera G A, et al. Scaling accurate genetic variant discovery to tens of thousands of samples [Internet]. 2017. doi:10.1101/201178
30. Garrison E, Marth G. Haplotype-based variant detection from short-read sequencing [Internet]. arXiv [q-bio.GN]. 2012. Available: arxiv.org/abs/1207.3907
31. McKenna A, Hanna M, Banks E, Sivachenko A, Cibulskis K, Kernytsky A, et al. The Genome Analysis Toolkit: a MapReduce framework for analyzing next-generation DNA sequencing data. Genome Res. 2010; 20: 1297-1303.
32. Home|Integrative Genomics Viewer [Internet]. [cited 7 Sep. 2018]. Available: www.broadinstitute.org/igv
33. Hogan G J, Vysotskaia V S, Beauchamp K A, Seisenberger S, Grauman P V, Haas K R, et al. Validation of an Expanded Carrier Screen that Optimizes Sensitivity via Full-Exon Sequencing and Panel-wide Copy Number Variant Identification. Clin Chem. 2018; 64: 1063-1073.
34. Richards S, Aziz N, Bale S, Bick D, Das S, Gastier-Foster J, et al. Standards and guidelines for the interpretation of sequence variants: a joint consensus recommendation of the American College of Medical Genetics and Genomics and the Association for Molecular Pathology. Genet Med. 2015; 17: 405-424.
35. Salvatier J, Wiecki T V, Fonnesbeck C. Probabilistic programming in Python using PyMC3. PeerJ Comput Sci. PeerJ Inc.; 2016; 2: e55.
36. Dobin A, Davis C A, Schlesinger F, Drenkow J, Zaleski C, Jha S, et al. STAR: ultrafast universal RNA-seq aligner. Bioinformatics. 2013; 29: 15-21.
37. Clopper C J, Pearson E S. The Use of Confidence or Fiducial Limits Illustrated in the Case of the Binomial. Biometrika. 1934; 26: 404.
38. Zook J M, Catoe D, McDaniel J, Vang L, Spies N, Sidow A, et al. Extensive sequencing of seven human genomes to characterize benchmark reference materials. Sci Data. 2016; 3: 160025.
39. Zook J M, Chapman B, Wang J, Mittelman D, Hofmann O, Hide W, et al. Integrating human sequence data sets provides a resource of benchmark SNP and indel genotype calls. Nat Biotechnol. 2014; 32: 246-251.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for detecting genetic variation in a genome of a subject, the genome comprising highly homologous first and second regions of interest, the method comprising:
(a) obtaining sequence reads by paired-end sequencing from multiple sites of interest in the first and second regions of interest, wherein the sequence reads comprise a first read and a second read obtained at each site of interest;
(b) aligning sequence reads to a reference genome, wherein first reads and second reads are aligned to the reference genome separately and the aligner emits multiple possible alignments for each of the first and second reads;
(c) identifying first reads and second reads that align to the first region of interest;
(d) pairing a first read and a second read from the reads identified in step (c), thereby generating a top paired alignment; and
(e) detecting the genetic variation in the top paired alignment generated in step (d).

2. The method of claim 1, comprising, before step (b), aligning first reads and second reads to a reference genome, wherein the aligner emits the best possible paired-end alignment to the first or second region of interest for each pair of first and second reads, and wherein only paired-end reads associated with a top alignment score to the first or second regions of interest are aligned separately in step (b).

3. The method of claim 1, wherein the sequence reads are obtained by direct targeted sequencing (DTS) of the multiple sites of interest, and wherein the first read comprises a genomic sequence read and the second read comprises a probe sequence read associated with a site of interest.

4. The method of claim 1, wherein in step (b) the sequence reads are aligned using the Burrows-Wheeler Aligner (BWA) algorithm.

5. The method of claim 1, wherein in step (b) the aligner only emits alignments that meet a minimum alignment score for the first and second regions of interest.

6. The method of claim 1, wherein a first read and a second read are paired in step (d) only if the alignments of the first read and the second read to the first region of interest are within a certain number of bases of each other.

7. The method of claim 1, wherein a first read and a second read are paired in step (d) only if the alignments of the first read and the second read to the first region of interest are within about 100 bp, about 200 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp, about 1500 bp, or more than 1500 bp.

8. The method of claim 1, comprising generating multiple paired alignments in step (d), calculating an alignment score for each of the multiple paired alignments, and identifying the top paired alignment as having the highest alignment score.

9. The method of claim 1, wherein the top paired alignment in step (d) is selected as having the smallest template length.

10-13. (canceled)

14. The method of claim 1, wherein the detecting in step (e) is based on an expected ploidy of 4.

15. The method of claim 1, wherein if a genetic variation is detected in step (e), a portion of the subject's genome is amplified by long-range PCR and assayed by multiplex ligation-dependent probe amplification (MLPA).

16. (canceled)

17. The method of claim 1, wherein if a genetic variation is detected in step (e), the subject's genomic DNA is assayed by multiplex ligation-dependent probe amplification (MLPA).

18-24. (canceled)

25. The method of claim 1, wherein the first region of interest comprises a gene and the second region of interest comprises a pseudogene.

26-28. (canceled)

29. The method according to claim 25, wherein the gene is PMS2.

30. The method according to claim 25, wherein the pseudogene is PMS2CL.

31. The method of claim 1, wherein the multiple sites of interest are within an exon of PMS2 and an exon in another part of the subject's genome.

32. The method of claim 1, wherein the multiple sites of interest are within an exon of PMS2 and an exon of PMS2CL.

33. The method of claim 1, wherein the multiple sites of interest are within exons 11, 12, 13, 14, and/or 15 of PMS2 and exons 2, 3, 4, 5, and/or 6 of PMS2CL.

34-36. (canceled)

37. A non-transitory computer-readable storage medium comprising computer-executable instructions for carrying out claim 1.

38. A system comprising:
(a) one or more processors;
(b) memory; and
(c) one or more programs, wherein the one or more programs are stored in the memory and configured to be executed by the one or more processors, the one or more programs including instructions for carrying out claim 1.
